(19) United States
(12) Patent Application Publication
(10) Pub. No.: US 2009/0076049 A1
(43) Pub. Date: Mar. 19, 2009

(54) Novel Spiro [Imidazolidine-4, 3' -Indole] 2, 2', 5' (1H) Triones for Treatment of Conditions Associated with Vanilloid Receptor 1

(75) Inventors: Lucy Horoszok, Sodertalje (SE); Carmen Leung, Montreal (CA); Miroslaw Tomaszewski, Montreal (CA); Christopher Walpole, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/278,663

(22) PCT Filed: Feb. 6, 2007
(86) PCT No.: PCT/SE07/00108

§ 371 Date: Aug. 20, 2008

Related U.S. Application Data
(60) Provisional application No. 60/771,159, filed on Feb. 7, 2006.

(51) Int. Cl. A61K 31/4188; C07D 487/10; A61K 31/4439; A61K 31/427; A61K 31/4245

(52) U.S. Cl. 514/278; 548/301.1; 546/15; 548/147; 548/143; 514/387; 514/365; 514/364

(57) ABSTRACT

The present invention relates to new compounds of formula (I), wherein R¹ to R⁹ and X are as defined as in formula I, or salts, solvates or solvated salts thereof, processes for their preparation and to new intermediates used in the preparation thereof, pharmaceutical formulations containing said compounds and to the use of said compounds in therapy.

### FIELD OF THE INVENTION

The present invention relates to new compounds, to pharmaceutical formulations containing said compounds and to the use of said compounds in therapy. The present invention further relates to processes for the preparation of said compounds and to the use of intermediates in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., Schumacher, M. A., et. al. Nature (1997) v. 389 p 816-824). Functional studies using VR1 indicate that it is also activated by noxious heat, tissue acidification and other inflammatory mediators (Tominaga, M., Caterina, M. J. et. al. Neuron (1998) v. 21, p. 531-543). Expression of VR1 is also regulated after peripheral nerve-damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects.

Compounds with VR1 inhibitor activity are believed to be of potential use for the treatment and/or prophylaxis of disorders such as pain, especially that of inflammatory or traumatic origin such as arthritis, ischaemia, cancer, fibromyalgia, low back pain and post-operative pain (Walker et al J Pharmacol Exp Ther. (2003) Jan; 304(1):56-62). In addition to this visceral pains such as chronic pelvic pain, cystitis, irritable bowel syndrome (IBS), pancreatitis and the like, as well as neuropathic pain such as sciatia, diabetic neuropathy, HIV neuropathy, multiple sclerosis, and the like (Walker et al ibid, Rashid et al J Pharmacol Exp Ther. (2003) March; 304(3):940-8), are potential pain states that could be treated with VR1 inhibiton. These compounds are also believed to be potentially useful for inflammatory disorders like asthma, cough, inflammatory bowel disease (IBD) (Hwang and Oh Curr Opin Pharmacol (2002) Jun; 2(3):235-42). Compounds with VR1 blocker activity are also useful for itch and skin diseases like psoriasis and for gastro-esophageal reflux disease (GERD), emesis, cancer, urinary incontinence and hyperactive bladder (Yiangou et al BJU Int (2001) Jun; 87(9):774-9, Szallasi Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid).

A further portential use relates to the treatment of tolerance to VR1 activators.

VR1 inhibitors may also be useful in the treatment of interstitial cystitis and pain related to interstitial cystitis.

VR1 inhibitors may also be useful in the treatment of obesity and migraine; WO2006/007851 discloses the use of VR1 antagonists for the treatment of obesity.

EP 66378 discloses biodegradable spiro-hydantoin derivatives for use as inhibitors of aldose reductase.

WO 92/07830 describes spiro-hydantoin derivatives and their use as antagonists for gastrin releasing peptide.

### DETAILED DESCRIPTION OF THE INVENTION

The object of the present invention is to provide compounds exhibiting an inhibitory activity at the vanilloid receptor 1 (VR1).

The first embodiment of the invention provides compounds of formula I

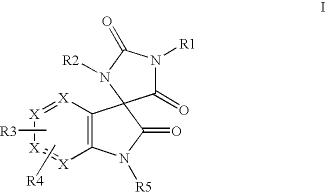

wherein:

R¹ is selected from H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, C₃₋₅heteroaryl, C₆₋₁₀aryl and C₃₋₆heterocycloalkyl, C₃₋₆heteroaryl-C₁₋₆alkyl, C₆₋₁₀aryl-C₁₋₆alkyl and C₁₋₆ alkyl-oxy-C₁₋₅alkyl, whereby R¹ may optionally be substituted by one or more groups selected from halogen, cyano, nitro, methoxy, ethoxy, methyl, ethyl, hydroxy and —NR⁶R⁷;

R² is selected from H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, C₃₋₅heteroaryl, C₆₋₁₀aryl, C₃₋₆heterocycloalkyl, C₃₋₆heteroaryl-C₁₋₆alkyl, C₆₋₁₀aryl-C₁₋₆alkyl and C₁₋₆ alkyl-oxy-C₁₋₅alkyl, whereby R² may optionally be substituted by one or more groups selected from halogen, cyano, nitro, methoxy, ethoxy, methyl, ethyl, hydroxy and —NR⁶R⁷;

R³ is selected from H, halogen, C₁₋₁₀alkyl, haloalkyl, haloalkylO, C₂₋₁₀alkenyl, C₂₋₁₀alkynyl, C₃₋₁₀cycloalkyl, C₃₋₁₀cycloalkyl-C₁₋₆alkyl and C₄₋₈cycloalkenyl-C₁₋₆alkyl;

R⁴ is selected from H, halogen, haloalkyl, haloalkylO, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl and C₄₋₈cycloalkenyl-C₁₋₆alkyl;

R⁵ is selected from C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocycloalkyl-C₁₋₆alkyl, C₃₋₅heteroaryl, C₃₋₆heterocycloalkyl, C₁₋₆ alkyl-oxy-C₁₋₅alkyl, C₂₋₆alkenyl-oxy-C₁₋₆alkyl, C₂₋₆ alkynyl-oxy-C₁₋₆alkyl, C₆₋₁₀aryl-oxy-C₁₋₆ alkyl, C₁₋₆ alkyl-O—C₅₋₁₀heteroaryl, C₆₋₁₀arylC₁₋₆alkyl, C₃₋₆heteroaryl-C₁₋₆alkyl, C₆₋₁₀aryl-C₂₋₆alkenyl, C₆₋₁₀aryl-C₂₋₆alkynyl, C₃₋₆heteroaryl-C₂₋₆alkenyl, C₃₋₆heteroaryl-C₂₋₆alkynyl, R⁶C(═O)N(—R⁷)—C₁₋₆alkyl, R⁶R⁷N—, R⁶R⁷N—C(═O)—C₁₋₆alkyl, R⁶R⁷NS(═O)₂—C₁₋₆alkyl, R⁶CS(═O)₂N(—R⁶)—C₁₋₆alkyl, R⁶R⁷NC(═O)N(—R⁸)—C₁₋₆alkyl, R⁶R⁷NC(═O)N(—R⁸)—C₁₋₆alkyl and R⁶R⁷NS(═O)₂N(R⁸)—C₁₋₆alkyl,

whereby any C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocycloalkyl-C₁₋₆alkyl, R⁶R⁷N—, C₃₋₅heteroaryl, C₆₋₁₀aryl, C₃₋₆heterocycloalkyl, C₁₋₆ alkyl-oxy-C₁₋₅alkyl, C₂₋₆ alkenyl-oxy-C₁₋₆alkyl, C₂₋₆ alkynyl-oxy-C₁₋₆alkyl, C₁₋₆ alkyl-oxy-C₆₋₁₀aryl, C₁₋₆ alkyl-oxy-C₅₋₁₀heteroaryl or C₆₋₁₀aryl-C₁₋₆alkyl, C₅₋₁₀heteroaryl-C₁₋₆alkyl, C₆₋₁₀aryl-C₂₋₆alkenyl, C₆₋₁₀aryl-C₂₋₆alkynyl, C₅₋₁₀heteroaryl-C₂₋₆alkenyl, C₆₋₁₀heteroaryl-C₂₋₆alkynyl, R⁶C(═O)N(—R⁷)—C₁₋₆alkyl and R⁶R⁷N—C(═O)—C₁₋₆alkyl, may optionally be substituted by one or more groups selected from halogen, cyano, nitro, CF₃, OCF₃, trimethylsilyl, hydroxy, —NR⁶R⁷, SO₂R⁷, R⁶O—C₁₋₆ alkyl, C₁₋₆alkyl, C₂₋₆alkenyl, C₂₋₆alkynyl, C₆₋₁₀aryl and C₅₋₁₀heteroaryl;

R⁶, R⁷ and R³ are independently selected from H, C₁₋₆alkyl, C₂₋₆alkenyl, C₂₋₆alkynyl, substituted or unsubstituted C₆₋₁₀aryl, substituted or unsubstituted C₃₋₆heteroaryl and a divalent C₁₋₆group that together with another divalent R⁵, R⁶ or R⁷ forms a portion of a ring;

X is selected from N, CH and CR⁹,

whereby R⁹ is selected from H, halogen, haloalkyl, haloalkylO, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;
or salts, solvates or solvated salts thereof,
with the proviso that R⁵ is not a naphthylmethyl or cinnamyl radical, and with the proviso that the compound does not have the formula III:

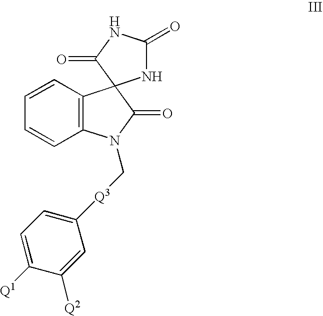

where Q¹ and Q² are independently halo or C₁₋₃haloalkyl and
Q³ is ethenyl or ethynyl.

A second embodiment of the invention relates to compounds according to formula I wherein:

R¹ is H, C₁₋₁₀alkyl or C₁₋₆ alkyl-oxy-C₁₋₅alkyl;

R² is H, C₁₋₁₀alkyl or C₁₋₆ alkyl-oxy-C₁₋₅alkyl;

R³ is H, halogen, C₁₋₁₀alkyl or haloalkylO;

R⁴ is H, halogen, haloalkylO or C₁₋₁₀alkyl;

R⁵ is C₂₋₁₀alkenyl, C₂₋₁₀alkynyl, C₃₋₁₀cycloalkyl, C₃₋₁₀cycloalkyl-C₁₋₆alkyl, C₃₋₆heterocycloalkyl-C₁₋₆alkyl, C₁₋₆ alkyl-oxy-C₁₋₅alkyl, C₆₋₁₀aryl-oxy-C₁₋₆ alkyl, C₆₋₁₀aryl-C₁₋₆alkyl, C₃₋₆heteroaryl-C₁₋₆alkyl, C₆₋₁₀aryl-C₂₋₆alkenyl, C₆₋₁₀aryl-C₂₋₆alkynyl, C₃₋₆heteroaryl-C₂₋₆alkenyl, C₃₋₆heteroaryl-C₂₋₆alkynyl or R⁶R⁷N—C(═O)—C₁₋₆alkyl, whereby any C₁₋₁₀allyl, C₃₋₁₀cycloalkyl, C₃₋₁₀cycloalkyl-C₁₋₆alkyl, C₃₋₆heterocycloalkyl-C₁₋₆alkyl, C₃₋₅heteroaryl, C₆₋₁₀aryl, C₅₋₁₀heteroaryl-C₁₋₆alkyl and R⁶R⁷N—C(═O)—C₁₋₆alkyl, may optionally be substituted by one or more groups selected from CF₃, methoxy, ethoxy, OCF₃, methyl, tert-butyl, SO₂R⁷, R⁶O—C₁₋₆ alkyl, C₁₋₆alkyl, C₂₋₆alkenyl, C₆₋₁₀aryl and C₅₋₁₀heteroaryl;

R⁶, R⁷ and R⁸ are independently selected from H, C₁₋₆alkyl, substituted or unsubstituted C₆₋₁₀aryl and substituted and unsubstituted C₃₋₆heteroaryl;

X is selected from N, CH and CR⁹,

wherein R⁹ is selected from H, halogen, haloalkylO and C₁₋₁₀alkyl.

Another embodiment of the invention relates to compounds of formula II,

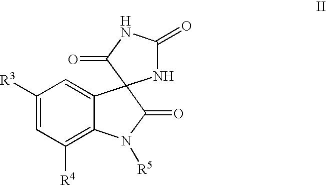

wherein R³ to R⁹ are as defined as in claims 1 or 2,
with the proviso that the compound does not have the formula III:

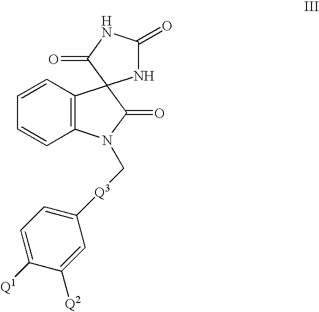

where Q¹ and Q² are independently halo or C₁₋₃haloalkyl and
Q³ is ethenyl or ethynyl.

A further embodiment of the invention relates to compounds of formula I or II wherein R³ is hydrogen, bromo, chloro, fluoro, methyl, ethyl, propyl or fluoromethyl, difluoromethyl, trifluoromethyl, fluoromethoxy, difluoromethoxy or trifluoromethoxy.

One embodiment relates to compounds of formula I or II wherein R³ is chloro.

Another embodiment of the invention relates to compounds of formula I or II wherein R³ is fluoro.

A further embodiment relates to compounds of formula I or II wherein R³ is methyl.

Yet another embodiment relates to compounds of formula I or II wherein R³ is hydrogen.

In a further embodiment R⁴ is hydrogen.

One embodiment of the invention relates to compounds of formula I or II wherein R³ is chloro and R⁴ is methyl.

In another embodiment R³ is substituted on position 5.

In a further embodiment R⁴ is substituted on position 7.

Another embodiment of the invention relates to compounds of I wherein

R¹ is hydrogen or methyl; and
R² is hydrogen or methyl.

In yet another embodiment R¹ and R² are methyl. In another embodiment R¹ and R² are hydrogen. In another embodiment R¹ is methyl and R² is hydrogen

One embodiment relates to compounds of formula I wherein X is CH.

In another embodiment of the invention R¹ is selected from the group comprising

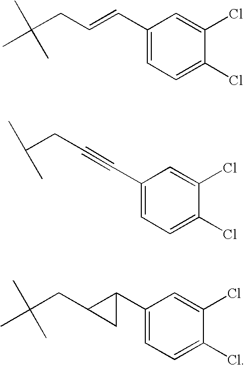

A further embodiment of the invention relates to compounds selected from the group consisting
1′-[(2,6-dichloro-4-pyridinyl)methyl]-2H,5H-spiro[imidazolidine-4,3′-indole]-2,2′,5(1′H))-trione,
1′-[2-(1H-indol-3-yl)ethyl]-2H,5H-spiro[imidazolidine-4,3′-indole]-2,2′,5(1′H)-trione,
1′-[3-(methyloxy)propyl]-2H,5H-spiro[imidazolidine-4,3′-indole]-2,2′,5(1′H)-trione,
1′-(cyclopropylmethyl)-2H,5H-spiro[imidazolidine-4,3′-indole]-2,2′,5(1′H)-trione,
1′-(2-{[2-(methyloxy)phenyl]oxy}ethyl)-2H,5H-spiro[imidazolidine-4,3′-indole]-2,2′,5(1′H)-trione,
1′-[(2-chloro-1,3-thiazol-5-yl)methyl]-2H,5H-spiro[imidazolidine-4,31-indole]-2,2′,5(1′H)-trione,
1′-{[5-(4-chlorophenyl)-1,3,4-oxadiazol-2-yl]methyl}-2H,5H-spiro[imidazolidine-4,3′-indole]-2,2′,5(1′H)-trione,
1′-(cyclohexylmethyl)-2H,5H-spiro[imidazolidine-4,3′-indole]-2,2′,5(1′H)-trione,
1′-[2-(methyloxy)ethyl]-2H,5H-spiro[imidazolidine-4,3′-indole]-2,2′,5(1′H)-trione,
5′-chloro-1′-(2-phenylethyl)-2H,5H-spiro[imidazolidine-4,3′-indole]-2,2′,5(1′H)-trione,
5′-chloro-1′-(2-{[2-(methyloxy)phenyl]oxy}ethyl)-2H,5H-spiro[imidazolidine-4,3′-indole]-2,2′,5(1′H)-trione,
5′-chloro-1′-[(6-chloro-3-pyridinyl)methyl]-2H,5H-spiro[imidazolidine-4,3′-indole]-2,2′,5(1′H)-trione,
5′-fluoro-1′-(2-phenylethyl)-2H,5H-spiro[imidazolidine-4,3′-indole]-2,2′,5(1′H)-trione,
5′-fluoro-1′-{2-[(4-fluorophenyl)oxy]ethyl}-2H,5H-spiro[imidazolidine-4,3′-indole]-2,2′,5(1′H)-trione,
1′-[(2,6-dichloro-4-pyridinyl)methyl]-5′-fluoro-2H,5H-spiro[imidazolidine-4,3′-indole]-2,2′,5(1′H)-trione,
5′-fluoro-1′-[2-(1H-indol-3-yl)ethyl]-2H,5H-spiro[imidazolidine-4,3′-indole]-2,2′,5(1′H)-trione,
5′-fluoro-1′-{[6-(trifluoromethyl)-3-pyridinyl]methyl}-2H,5H-spiro[imidazolidine-4,3′-indole]-2,2′,5(1′H)-trione,
5′-fluoro-1′-[3-(methyloxy)propyl]-2H,5H-spiro[imidazolidine-4,3′-indole]-2,2′,5(1′H)-trione,
5′-fluoro-1′-{[5-(trifluoromethyl)-2-furanyl]methyl}-2H,5H-spiro[imidazolidine-4,3′-indole]-2,2′,5(1′H)-trione,
1′-(cyclopropylmethyl)-5′-fluoro-2H,5H-spiro[imidazolidine-4,3′-indole]-2,2′,5(1′H)-trione,
5′-fluoro-1′-[2-(phenyloxy)ethyl]-2H,5H-spiro[imidazolidine-4,3′-indole]-2,2′,5(1′H)-trione,
5′-fluoro-1′-[2-(1H-pyrrol-1-yl)ethyl]-2H,5H-spiro[imidazolidine-4,3′-indole]-2,2′,5(1′H)-trione,
5′-fluoro-1′-[(5-methyl-3-isoxazolyl)methyl]-2H,5H-spiro[imidazolidine-4,3′-indole]-2,2′,5(1′H)-trione,
5′-fluoro-1′-(2-{[2-(methyloxy)phenyl]oxy}ethyl)-2H,5H-spiro[imidazolidine-4,3′-indole]-2,2′,5(1′H)-trione,
1′-[(2-chloro-1,3-thiazol-5-yl)methyl]-5′-fluoro-2H,5H-spiro[imidazolidine-4,3′-indole]-2,2′,5(1′H)-trione,
5′-fluoro-1′-({5-[4-(methyloxy)phenyl]-1,3,4-oxadiazol-2-yl}methyl)-2H,5H-spiro[imidazolidine-4,3′-indole]-2,2′,5(1′H)-trione,
1′-(cyclohexylmethyl)-5′-fluoro-2H,5H-spiro[imidazolidine-4,3′-indole]-2,2′,5(1′H)-trione,
1′-[(6-chloro-3-pyridinyl)methyl]-5′-fluoro-2H,5H-spiro[imidazolidine-4,3′-indole]-2,2′,5(1′H)-trione,
5′-fluoro-1′-(2-propynyl)-2H,5H-spiro[imidazolidine-4,3′-indole]-2,2′,5(1′H)-trione,
1′-(cyclobutylmethyl)-5′-fluoro-2H,5H-spiro[imidazolidine-4,3′-indole]-2,2′,5(1′H)-trione,
1′-[2-(ethyloxy)ethyl]-5′-fluoro-2H,5H-spiro[imidazolidine-4,3′-indole]-2,2′,5(1′H)-trione,
5′-fluoro-1′-[2-(methyloxy)ethyl]-2H,5H-spiro[imidazolidine-4,3′-indole]-2,2′,5(1′H)-trione,
1′-[(3,5-dimethyl-4-isoxazolyl)methyl]-5′-fluoro-2H,5H-spiro[imidazolidine-4,3′-indole]-2,2′,5(1′H)-trione,
5′-chloro-1′-{2-[(4-fluorophenyl)oxy]ethyl}-7′-methyl-2H,5H-spiro[imidazolidine-4,3′-indole]-2,2′,5(1′H)-trione,
5′-chloro-7′-methyl-1′-[2-(1H-pyrrol-1-yl)ethyl]-2H,5H-spiro[imidazolidine-4,3′-indole]-2,2′,5(1′H)-trione,
5′-chloro-1′-(imidazo[1,2-a]pyridin-2-ylmethyl)-7′-methyl-2H,5H-spiro[imidazolidine-4,3′-indole]-2,2′,5 (1′H)-trione,
5′-chloro-1′-(cyclohexylmethyl)-7′-methyl-2H,5H-spiro[imidazolidine-4,3′-indole]-2,2′,5(1′H)-trione,
5′-chloro-1′-[(6-chloro-3-pyridinyl)methyl]-7′-methyl-2H,5H-spiro[imidazolidine-4,3′-indole]-2,2′,5(1′H)-trione,
5′-chloro-1′-(cyclobutylmethyl)-7′-methyl-2H,5H-spiro[imidazolidine-4,3′-indole]-2,2′,5(1′H)-trione,
5′-chloro-1′-[2-(ethyloxy)ethyl]-7′-methyl-2H,5H-spiro[imidazolidine-4,3′-indole]-2,2′,5(1′H)-trione,
5′-chloro-7′-methyl-1′-[2-(methyloxy)ethyl]-2H,5H-spiro[imidazolidine-4,3′-indole]-2,2′,5(1′H)-trione,
5′-chloro-1′-[(3,5-dimethyl-4-isoxazolyl)methyl]-7′-methyl-2H,5H-spiro[imidazolidine-4,3′-indole]-2,2′,5(1′H)-trione,
1′-(2,1,3-benzoxadiazol-5-ylmethyl)-2H,5H-spiro[imidazolidine-4,3′-indole]-2,2′,5(1′H)-trione,
1′-[2-(1H-pyrrol-1-yl)ethyl]-2H,5H-spiro[imidazolidine-4,3′-indole]-2,2′,5(1′H)-trione,
1′-[(6-chloro-3-pyridinyl)methyl]-2H,5H-spiro[imidazolidine-4,3′-indole]-2,2′,5(1′H)-trione,
1′-pentyl-2H,5H-spiro[imidazolidine-4,3′-indole]-2,2′,5(1′H)-trione,
1′-(cyclobutylmethyl)-2H,5H-spiro[imidazolidine-4,3′-indole]-2,2′,5(1′H)-trione,
1′-[2-(ethyloxy)ethyl]-2H,5H-spiro[imidazolidine-4,3′-indole]-2,2′,5(1′H)-trione,
1′-[(3,5-dimethyl-4-isoxazolyl)methyl]-2H,5H-spiro[imidazolidine-4,3′-indole]-2,2′,5(1′H)-trione,
1′-{2-[(4-fluorophenyl)oxy]ethyl}-5′-methyl-2H,5H-spiro[imidazolidine-4,3′-indole]-2,2′,5(1′H)-trione,
1′-(2,1,3-benzoxadiazol-5-ylmethyl)-5′-methyl-2H,5H-spiro[imidazolidine-4,3′-indole]-2,2′,5(1′H)-trione,
5′-methyl-1′-{[6-(trifluoromethyl)-3-pyridinyl]methyl}-2H,5H-spiro[imidazolidine-4,3′-indole]-2,2′,5(1′H)-trione,
5′-methyl-1′-[3-(methyloxy)propyl]-2H,5H-spiro[imidazolidine-4,3′-indole]-2,2′,5(1′H)-trione,
5′-methyl-1′-[2-(1H-pyrrol-1-yl)ethyl]-2H,5H-spiro[imidazolidine-4,3′-indole]-2,2′,5(1′H)-trione,
1′-[(6-chloro-3-pyridinyl)methyl]-5′-methyl-2H,5H-spiro[imidazolidine-4,3′-indole]-2,2′,5(1′H)-trione,
5′-methyl-1′-[2-(methyloxy)ethyl]-2H,5H-spiro[imidazolidine-4,3′-indole]-2,2′,5(1′H)-trione,
1′-[(3,5-dimethyl-4-isoxazolyl)methyl]-5′-methyl-2H,5H-spiro[imidazolidine-4,3′-indole]-2,2′,5(1′H)-trione,
5′-methyl-1′-(1-phenylethyl)-2H,5H-spiro[imidazolidine-4,3′-indole]-2,2′,5(1′H)-trione,
5′-methyl-1′-(2-phenylethyl)-2H,5H-spiro[imidazolidine-4,3′-indole]-2,2′,5(1′H)-trione,
1′-[2-(1H-indol-3-yl)ethyl]-5′-methyl-2H,5H-spiro[imidazolidine-4,3′-indole]-2,2′,5(1′H)-trione,
5′-methyl-1′-{[5-(trifluoromethyl)-2-furanyl]methyl}-2H,5H-spiro[imidazolidine-4,3′-indole]-2,2′,5(1′H)-trione,
1′-(cyclopropylmethyl)-5′-methyl-2H,5H-spiro[imidazolidine-4,3′-indole]-2,2′,5(1′H)-trione,
5′-methyl-1′-[2-(phenyloxy)ethyl]-2H,5H-spiro[imidazolidine-4,3′-indole]-2,2′,5(1′H)-trione,
5′-methyl-1′-[(5-methyl-3-isoxazolyl)methyl]-2H,5H-spiro[imidazolidine-4,3′-indole]-2,2′,5(1′H)-trione,
5′-methyl-1′-(2-{[2-(methyloxy)phenyl]oxy}ethyl)-2H,5H-spiro[imidazolidine-4,3′-indole]-2,2′,5(1′H)-trione,
1′-[(2-chloro-1,3-thiazol-5-yl)methyl]-5′-methyl-2H,5H-spiro[imidazolidine-4,3′-indole]-2,2′,5(1′H)-trione,
1′-{[5-(4-chlorophenyl)-1,3,4-oxadiazol-2-yl]methyl}-5′-methyl-2H,5H-spiro[imidazolidine-4,3′-indole]-2,2′,5(1′H)-trione,
5′-methyl-1′-({5-[4-(methyloxy)phenyl]-1,3,4-oxadiazol-2-yl}methyl)-2H,5H-spiro[imidazolidine-4,3′-indole]-2,2′,5(1′H)-trione,
1′-(cyclohexylmethyl)-5′-methyl-2H,5H-spiro[imidazolidine-4,3′-indole]-2,2′,5(1′H)-trione,
1′-(cyclobutylmethyl)-5′-methyl-2H,5H-spiro[imidazolidine-4,3′-indole]-2,2′,5(1′H)-trione,
1′-[2-(ethyloxy)ethyl]-5′-methyl-2H,5H-spiro[imidazolidine-4,3′-indole]-2,2′,5(1′H)-trione,
1′-(tetrahydro-2H-pyran-2-ylmethyl)-2H,5H-spiro[imidazolidine-4,3′-indole]-2,2′,5(1′H)-trione,
1′-(2-propynyl)-2H,5H-spiro[imidazolidine-4,3′-indole]-2,2′,5(1′H)-trione,
5′-chloro-1′-[2-(phenyloxy)ethyl]-2H,5H-spiro[imidazolidine-4,3′-indole]-2,2′,5(1′H)-trione,
5′-chloro-1′-[2-(1H-pyrrol-1-yl)ethyl]-2H,5H-spiro[imidazolidine-4,3′-indole]-2,2′,5(1′H)-trione,
5′-chloro-1′-[(2-chloro-1,3-thiazol-5-yl)methyl]-2H,5H-spiro[imidazolidine-4,3′-indole]-2,2′,5(1′H)-trione,
5′-chloro-1′-(cyclohexylmethyl)-2H,5H-spiro[imidazolidine-4,3′-indole]-2,2′,5(1′H)-trione,
5′-chloro-1′-(cyclobutylmethyl)-2H,5H-spiro[imidazolidine-4,3′-indole]-2,2′,5(1′H)-trione,
5′-chloro-1′-[2-(ethyloxy)ethyl]-2H,5H-spiro[imidazolidine-4,3′-indole]-2,2′,5(1′H)-trione,
5′-chloro-1′-[2-(methyloxy)ethyl]-2H,5H-spiro[imidazolidine-4,3′-indole]-2,2′,5(1′H)-trione,
5′-chloro-1′-[(3,5-dimethyl-4-isoxazolyl)methyl]-2H,5H-spiro[imidazolidine-4,3′-indole]-2,2′,5(1′H)-trione,
5′-fluoro-1′-(imidazo[1,2-a]pyridin-2-ylmethyl)-2H,5H-spiro[imidazolidine-4,3′-indole]-2,2′,5(1′H)-trione,
1′-{[5-(4-chlorophenyl)-1,3,4-oxadiazol-2-yl]methyl}-5′-fluoro-2H,5H-spiro[imidazolidine-4,3′-indole]-2,2′,5(1′H)-trione,
5′-fluoro-1′-(3-pyridinylmethyl)-2H,5H-spiro[imidazolidine-4,3′-indole]-2,2′,5(1′H)-trione,
5′-fluoro-1′-(tetrahydro-2H-pyran-2-ylmethyl)-2H,5H-spiro[imidazolidine-4,3′-indole]-2,2′,5(1′H)-trione,
1′-(2,1,3-benzoxadiazol-5-ylmethyl)-5′-fluoro-2H,5H-spiro[imidazolidine-4,3′-indole]-2,2′,5(1′H)-trione,
5′-chloro-7′-methyl-1′-(2-phenylethyl)-2H,5H-spiro[imidazolidine-4,3′-indole]-2,2′,5(1′H)-trione,
1′-(2,1,3-benzoxadiazol-5-ylmethyl)-5′-chloro-7′-methyl-2H,5H-spiro[imidazolidine-4,3′-indole]-2,2′,5(1′H)-trione,
5′-chloro-7′-methyl-1′-{[6-(trifluoromethyl)-3-pyridinyl]methyl}-2H,5H-spiro[imidazolidine-4,3′-indole]-2,2′,5(1′H)-trione,
5′-chloro-7′-methyl-1′-[(5-methyl-3-isoxazolyl)methyl]-2H,5H-spiro[imidazolidine-4,3′-indole]-2,2′,5(1′H)-trione,
5′-chloro-7′-methyl-1′-(2-{[2-(methyloxy)phenyl]oxy}ethyl)-2H,5H-spiro[imidazolidine-4,3′-indole]-2,2′,5(1′H)-trione,
5′-chloro-1′-[(2-chloro-1,3-thiazol-5-yl)methyl]-7′-methyl-2H,5H-spiro[imidazolidine-4,3′-indole]-2,2′,5(1′H)-trione,
1′-[(2,6-dichloro-4-pyridinyl)methyl]-5′-methyl-2H,5H-spiro[imidazolidine-4,3′-indole]-2,2′,5(1′H)-trione,
5′-methyl-1′-(tetrahydro-2H-pyran-2-ylmethyl)-2H,5H-spiro[imidazolidine-4,3′-indole]-2,2′,5(1′H)-trione,
5′-methyl-1′-(2-propynyl)-2H,5H-spiro[imidazolidine-4,3′-indole]-2,2′,5(1′H)-trione,
1′-(imidazo[1,2-a]pyridin-2-ylmethyl)-5′-methyl-2H,5H-spiro[imidazolidine-4,3′-indole]-2,2′,5(1′H)-trione,
   or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having is m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “aryl” used alone or as suffix or prefix, refers to a hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring. Said heteroaryl may be substituted or unsubstituted.

The term “non-aromatic group” or “non-aromatic” used alone, as suffix or as prefix, refers to a chemical group or radical that does not contain a ring having aromatic character (e.g., 4n+2 delocalized electrons).

The term “heteroalkyl” used alone or as a suffix or prefix, refers to a radical formed as a result of replacing one or more carbon atom of an alkyl with one or more heteroatoms selected from N, O, P and S.

The term “heteroaromatic” used alone or as a suffix or prefix, refers to a ring-containing structure or molecule having one or more multivalent heteroatoms, independently selected from N, O, P and S, as a part of the ring structure and including at least 3 and up to is about 20 atoms in the ring(s), wherein the ring-containing structure or molecule has an aromatic character (e.g., 4n+2 delocalized electrons).

The term “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a radical derived from a heterocycle by removing at least one hydrogen from a carbon of a ring of the heterocycle.

The term “heteroaryl” used alone or as a suffix or prefix, refers to a heterocyclyl having aromatic character, wherein the radical of the heterocyclyl is located on either a carbon or a heteroatom of an aromatic ring of the heterocyclyl. Said heteroaryl may be substituted or unsubstituted.

The term “heterocycloalkyl” used alone or as a suffix or prefix, refers to a heterocyclyl that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein_one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₁₂hydrocarbyl. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, firazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more is than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein -R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “aryloxy” used alone or as suffix or prefix, refers to radicals of the general formula —O—Ar, wherein —Ar is an aryl.

The term “heteroaryloxy” used alone or as suffix or prefix, refers to radicals of the general formula —O—Ar′, wherein —Ar′ is a heteroaryl.

The term “amine” or “amino” used alone or as a suffix or prefix, refers to radicals of the general formula —NRR′, wherein R and R′ are independently selected from hydrogen or a hydrocarbon radical.

“Halogen” includes fluorine, chlorine, bromine and iodine.

“Halogenated,” used as a prefix of a group, means one or more hydrogens on the group is replaced with one or more halogens.

“RT” or “rt” means room temperature.

“Saturated carbon” means a carbon atom in a structure, molecule or group wherein all the bonds connected to this carbon atom are single bond. In other words, there is no double or triple bonds connected to this carbon atom and this carbon atom generally adopts an sp³ atomic orbital hybridization.

“Unsaturated carbon” means a carbon atom in a structure, molecule or group wherein at least one bond connected to this carbon atom is not a single bond. In other words, there is at least one double or triple bond connected to this carbon atom and this carbon atom generally adopts a sp or sp² atomic orbital hybridization.

The term ‘a divalent C₁₋₆group that together with another divalent R⁵, R⁶ or R⁷ forms a portion of a ring’ means that R⁵, R⁶ or R⁷ can be cyclic e.g.

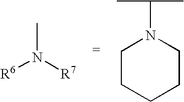

4, 5, 6, 7 membered rings with and without heteroatoms (O,N).

The present invention relates to the compounds of formula I as hereinbefore defined as well as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

### Methods of Preparation

One embodiment of the present invention provides processes for preparing compounds of formula I, or salts, solvates or solvated salts thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). For representative examples of heterocyclic chemistry see for example “Heterocyclic Chemistry”, J. A. Joule, K. Mills, G. F. Smith, 3^{rd} ed. Chapman and Hall (1995), p. 189-224 and “Heterocyclic Chemistry”, T. L. Gilchrist, 2^{nd} ed. Longman Scientific and Technical (1992), p. 248-282.

The term “room temperature” and “ambient temperature” shall mean, unless otherwise specified, a temperature between 16 and 25° C.

### Schemes

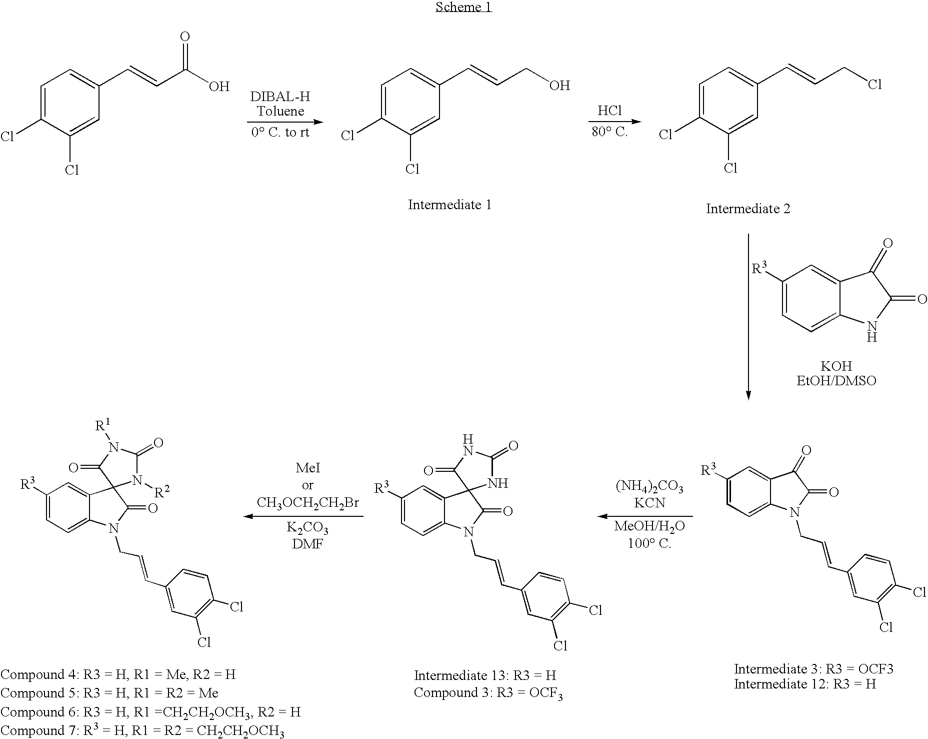

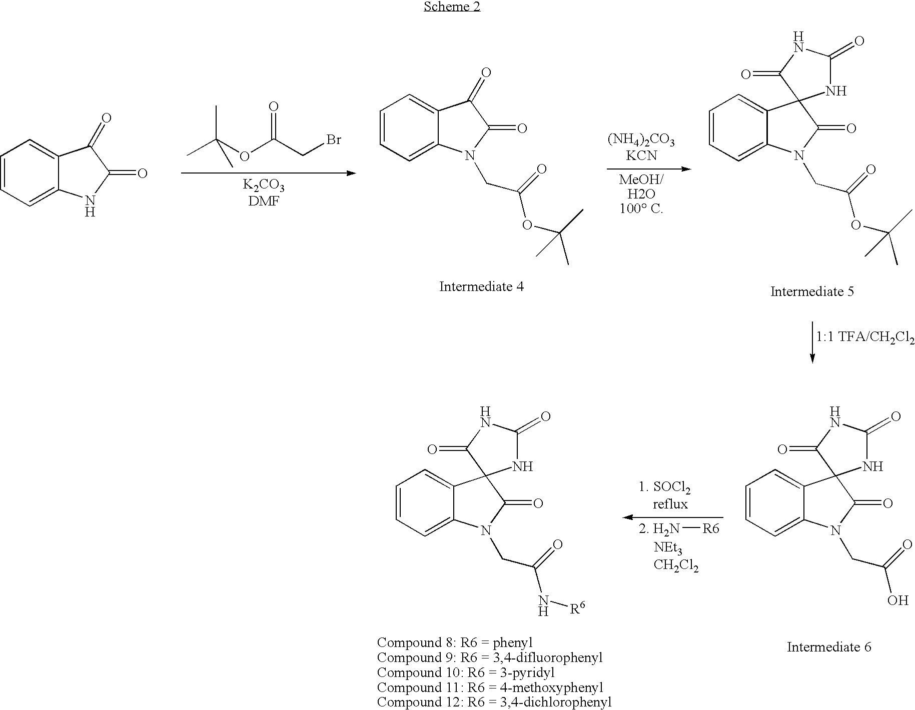

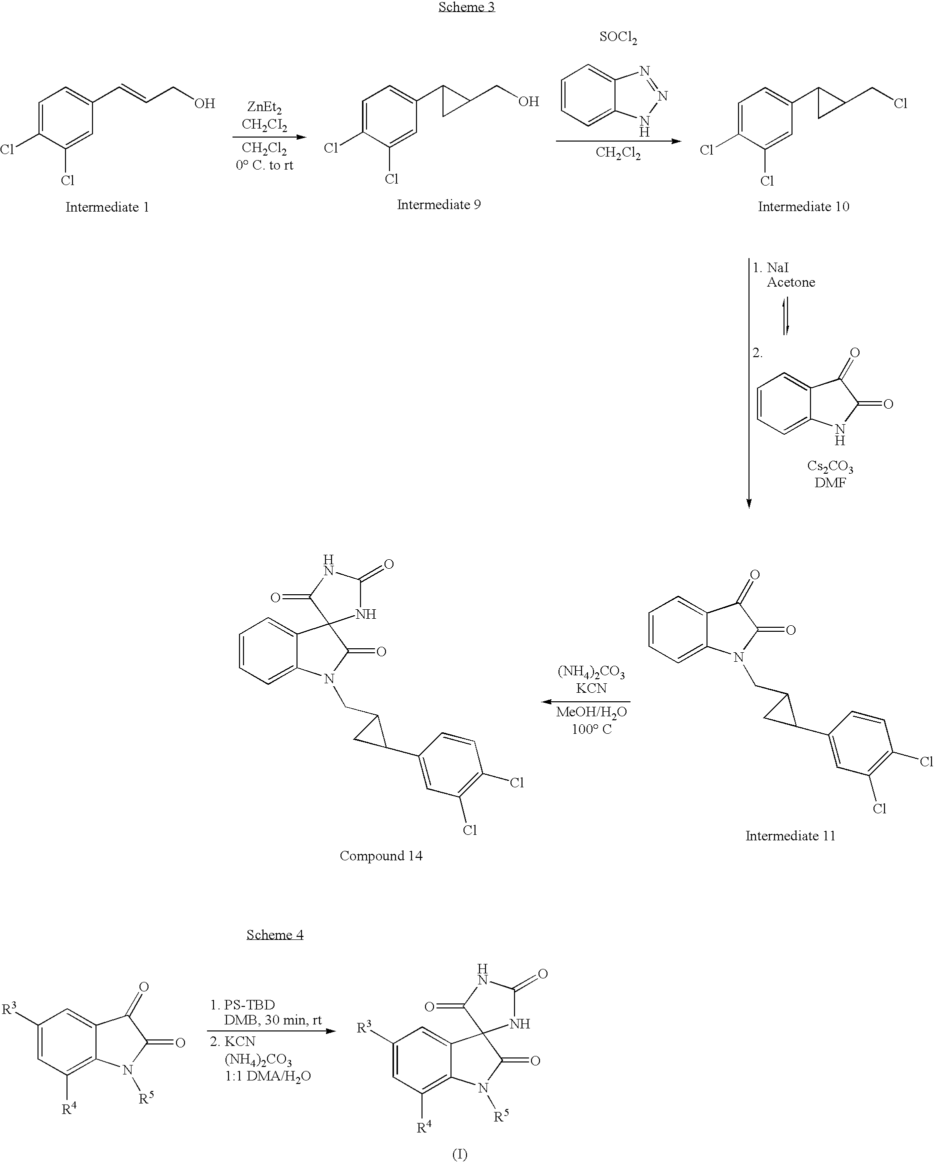

One embodiment of the invention relates to a process for the preparation of compounds of formula I, wherein R¹ to R⁹ and X are as defined as hereinabove, comprising:

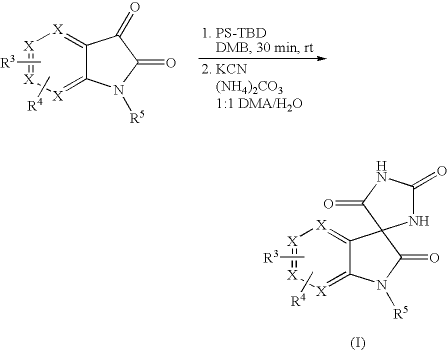

### INTERMEDIATES

A further embodiment of the invention relates to compounds selected from the group consisting of
(2E)-3-(3,4-dichlorophenyl)prop-2-en-1-ol,
1,2-dichloro-4-[(1E)-3-chloroprop-1-en-1-yl]benzene,
1-[(2E)-3-(3,4-dichlorophenyl)prop-2-en-1-yl]-5-(trifluoromethoxy)-1H-indole-2,3-dione,
tert-butyl (2,3-dioxo-2,3-dihydro-1H-indol-1-yl)acetate,
tert-butyl (2,2′,5-trioxospiro[imidazolidine-4,3′-indol]-1′(2′H)-yl)acetate,
(2,2′,5-trioxospiro[imidazolidine-4,3′-indol]-1′(2′H)-yl)acetic acid,
[2-(3,4-dichlorophenyl)cyclopropyl]methanol,
1,2-dichloro-4-[2-(chloromethyl)cyclopropyl]benzene,
1-{[2-(3,4-dichlorophenyl)cyclopropyl]methyl}-1H-indole-2,3-dione,
1-[(2E)-3-(3,4-dichlorophenyl)prop-2-en-1-yl]-1H-indole-2,3-dione, and
1′-[(2E)-3-(3,4-dichlorophenyl)prop-2-en-1-yl]-2H,5H-spiro[imidazolidine-4,3′-indole]-2,2′,5(1′H)-trione,
   which may be used as intermediates in the preparation of compounds suited for the treatment of VR1 mediated disorders, especially for use as intermediates for the preparation of compounds of formula I.

A yet further embodiment of the invention relates to compounds selected from the group consisting of
1-[(2E)-3-(3,4-dichlorophenyl)prop-2-en-1-yl]-1H-indole-2,3-dione,
1-{[2-(3,4-dichlorophenyl)cyclopropyl]methyl}-1H-indole-2,3-dione, and
(2,2′,5-trioxospiro[imidazolidine-4,3′-indol]-1′(2′H)-yl)acetic acid,
   which may be used as intermediates in the preparation of compounds suited for the treatment of VR1 mediated disorders, especially for use as intermediates for the preparation of compounds of formula I.

### Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream, for rectal administration e.g. as a suppository or for inhalation.

In general the above compositions may be prepared in a conventional manner using one or more conventional excipients, pharmaceutical acceptable diluents and/or inert carriers.

Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Medical Use

The compounds according to the present invention are useful in therapy. The compounds of the invention, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed in the peripheral nervous system and in other tissues. Thus, it is expected that the compounds of the invention are well suited for the treatment of VR1 mediated disorders.

The compounds of the invention are expected to be suitable for the treatment of acute and chronic pain, acute and chronic neuropathic pain and acute and chronic inflammatory pain.

Examples of such disorder may be selected from the group comprising low back pain, post-operative pain, visceral pains like chronic pelvic pain and the like.

The compounds of the invention are also expected to be suitable for the treatment of acute and chronic nociceptive pain.

Further relevant disorders may be selected from the group comprising cystitis, including interstitial cystitis and pain related thereto, ischeamic, sciatia, diabetic neuropathy, multiple sclerosis, arthritis, osteoarthritis, rheumatoid arthritis, fibromyalgia, pain and other signs and symptoms associated with psoriasis, pain and other signs and symptoms associated with cancer, emesis, urinary incontinence, hyperactive bladder and HIV neuropathy.

Additional relevant disorders may be selected from the group comprising gastro-esophageal reflux disease (GERD), irritable bowel syndrome (IBS), inflammatory bowel disease (IBD) and pancreatitis.

Other relevant disorders are related to respiratory diseases and may be selected from the group comprising asthma, cough, chronic obstructive lung disease, specifically chronic obstructive pulmonary disease (COPD) and emphysema, lung fibrosis and interstitial lung disease.

Yet other relevant disorders are obesity and obesity-related diseases or disorders, and migraine.

In one embodiment the obesity or obesity-related diseases or disorders is selected from the following: cardiovascular disease, hypertension, cancer and reproductive disorders.

The VR1 inhibitor(s) may be administrated by either an oral or inhaled route. The respiratory disease may be an acute and chronic illness and may be related to infection(s) and/or exposure to environmental pollution and/or irritants.

The compounds of the invention may also be used as antitoxin to treat (over-) exposure to VR1 activators like capsaicin, tear gas, acids or heat. Regarding heat, there is a potential use for VR1 antagonists in (sun-) burn induced pain, or inflammatory pain resulting from burn injuries.

The compounds may further be used for treatment of tolerance to VR1 activators.

One embodiment of the invention relates to the compounds of the invention as hereinbefore defined, for use as a medicament.

Another embodiment of the invention relates to the compounds of the invention as hereinbefore defined, for use as a medicament for treatment of VR1 mediated disorders.

A further embodiment of the invention relates to the compounds of the invention as hereinbefore defined, for use as a medicament for treatment of acute and chronic pain disorders.

Yet another embodiment of the invention relates to the compounds of the invention as hereinbefore defined, for use as a medicament for treatment of acute and chronic nociceptive pain.

Yet another embodiment of the invention relates to the compounds of the invention as hereinbefore defined, for use as a medicament for treatment of acute and chronic neuropathic pain.

Yet a further embodiment of the invention relates to the compounds of the invention as hereinbefore defined, for use as a medicament for treatment of acute and chronic inflammatory pain.

One embodiment of the invention relates to the compounds of the invention as hereinbefore defined, for use as a medicament for treatment of low back pain, post-operative pain and visceral pains like chronic pelvic pain.

Another embodiment of the invention relates to the compounds of the invention as hereinbefore defined, for use as medicaments for treatment of cystitis, including interstitial cystitis and pain related thereto, ischeamic, sciatia, diabetic neuropathy, multiple sclerosis, arthritis, osteoarthritis, rheumatoid arthritis, fibromyalgia, pain and other signs and symptoms associated with psoriasis, pain and other signs and symptoms associated with cancer, emesis, urinary incontinence, hyperactive bladder and HIV neuropathy.

A further embodiment of the invention relates to the compounds of the invention as hereinbefore defined, for use as a medicament for treatment of gastro-esophageal reflux disease (GERD), irritable bowel syndrome (IBS), inflammatory bowel disease (IBD) and pancreatitis.

Yet a further embodiment of the invention relates to the compounds of the invention as hereinbefore defined, for use as a medicament for treatment of respiratory diseases selected from the group comprising asthma, cough, chronic obstructive pulmonary disease (COPD), chronic obstructive lung disease and emphysema, lung fibrosis and interstitial lung disease.

One embodiment of the invention relates to the use of the compound of the invention as hereinbefore defined, in the manufacture of a medicament for treatment of VR1 mediated disorders and for treatment of acute and chronic pain disorders, acute and chronic neuropathic pain and acute and chronic inflammatory pain, and respiratory diseases and any other disorder mentioned above.

Another embodiment of the invention relates to a method of treatment of VR1 mediated disorders and acute and chronic pain disorders, acute and chronic neuropathic pain and acute and chronic inflammatory pain, and respiratory diseases, and any other disorder mentioned above, comprising administering to a mammal, including man in need of such treatment, a therapeutically effective amount of the compounds of the invention, as hereinbefore defined.

A further embodiment of the invention relates to a pharmaceutical composition comprising a compound of the invention as hereinbefore defined, for use in treatment of VR1 mediated disorders and for treatment of acute and chronic pain disorders, acute and chronic neuropathic pain and acute and chronic inflammatory pain, and respiratory diseases, and any other disorder mentioned above.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

### Non-Medical Use

In addition to their use in therapeutic medicine, the compounds of the invention, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and mice, as part of the search for new therapeutics agents.

### EXAMPLES

The invention will now be illustrated by the following non-limiting examples.

### General Methods

The invention will now be illustrated by the following Examples in which, generally:

(i) operations were carried out at ambient or room temperature, i.e. in the range 17 to 25° C. and under an atmosphere of an inert gas such as argon unless otherwise stated;

(ii) evaporations were carried out by rotary evaporation in vacuo and work-up procedures were carried out after removal of residual solids by filtration;

(iii) The ¹H NMR spectra were recorded on Brucker at 400 MHz. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD//ammonium acetate buffer) ionisation techniques;

(iv) yields, where present, are not necessarily the maximum attainable;

(v) the following abbreviations have been used:—
alloc allyloxycarbonyl
DCE dichloroethane
DCM dichloromethane
DMAP dimethylaminopyridine
EDC 1-(3-dimethylaminopropyl)-3-ethylcarbodiimide hydrochloride
HATU O-(7-azabenzotriazol-1-yl)-N,N,N′,N′-tetramethyluronium-hexafluorophosphate
HPLC high performance liquid chromatography
LC liquid chromatography
MsCl methanesulfonyl chloride
MS mass spectrometry
ret. time retention time
TFA trifluoroacetic acid
THF tetrahydrofurane
DMF dimethylormamide
TMEDA tetramethylethylenediamine
EtOAc ethyl acetate
BuLi Butyl lithium
TMEDA tetramethylethylenediamine

### Intermediate 1: (2E)-3-(3,4-dichlorophenyl)prop-2-en-1-ol

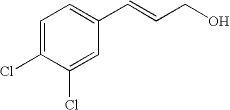

To a mixture of 3,4-dichlorocinnamic acid (2.00 g, 9.21 mmol) in toluene (46 mL) at 0° C. was added DIBAL-H (1.0 M solution in toluene, 24 mL, 23.96 mmol). The reaction gradually warmed up to room temperature and was stirred overnight. The reaction was then cooled to 0° C. and quenched with 5N HCl (8 mL). The reaction was diluted with EtOAc and washed with H₂O (2×). The aqueous layers were extracted with additional EtOAc (1×). The combined organic phases was dried over Na₂SO₄, filtered and concentrated in vacuo. The crude product was purified by silica gel column chromatography, eluting with 3:2 EtOAc:Hexanes, to give the title compound as a pale yellow solid (1.17 g, 63% yield). ¹H NMR (400 MHz, CDCl₃) δ 4.34 (dd, J=5.37, 1.66 Hz, 2H), 6.36 (dt, J=15.87, 5.35 Hz, 1H), 6.54 (dt, J=16.01, 1.46 Hz, 1H), 7.21 (dd, J=8.30, 2.05 Hz, 1H), 7.38 (d, J=8.40 Hz, 1H), 7.46 (d, J=2.15 Hz, 1H).

### Intermediate 2: 1,2-dichloro-4-[(1E)-3-chloroprop-1-en-1-yl]benzene

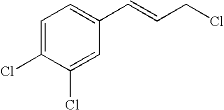

A mixture of (2E)-3-(3,4-dichlorophenyl)prop-2-en-1-ol (530 mg, 2.61 mmol) in concentrated HCl (4 mL) was heated at 80° C. for 3 hours. The reaction was then cooled, diluted with ether and washed with H₂O (3×). The aqueous layers were extracted with additional ether (1×). The combined organic phases was dried over Na₂SO₄, filtered and concentrated in vacuo. Further purification of the residue was not necessary. The title compound was obtained as a yellow oil (547 mg, 95% yield). ¹H NMR (400 MHz, CDCl₃) δ 4.22 (dd, J=7.03, 1.37 Hz, 2H), 6.32 (dt, J=15.67, 7.01 Hz, 1H), 6.57 (d, J=15.62 Hz, 1H), 7.21 (dd, J=8.40, 2.15 Hz, 1H), 7.40 (d, J=8.20 Hz, 1H), 7.47 (d, J=2.15 Hz, 1H).

### Intermediate 3: 1-[(2E)-3-(3,4-dichlorophenyl)prop-2-en-1-yl]-5-(trifluoromethoxy)-1H-indole-2,3-dione

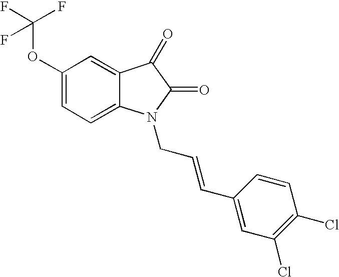

To a solution of 5-(trifluoromethoxy)isatin (197 mg, 0.85 mmol) in DMSO (2.0 mL) was added a solution of potassium hydroxide (48 mg, 0.85 mmol) in EtOH (1.0 mL). The is reaction was stirred at room temperature for 15 minutes and then 1,2-dichloro-4-[(1E)-3-chloroprop-1-en-1-yl]benzene (208 mg, 0.94 mmol) was added. The reaction was stirred at room temperature overnight, poured into H₂O and filtered. The precipitate was rinsed with H₂O, dissolved in CH₂Cl₂ and washed with H₂O (2×). The aqueous layers were extracted with additional CH₂Cl₂ (1×). The combined organic phases was dried over Na₂SO₄, filtered and concentrated in vacuo. The crude product was purified by silica gel column chromatography, eluting with 1:3 EtOAc:Hexanes, to give the title compound as an orange solid (181 mg, 51% yield). ¹H NMR (400 MHz, CDCl₃) δ 4.55 (dd, J=5.86, 1.56 Hz, 2H), 6.18 (dt, J=15.96, 5.98 Hz, 1H), 6.59 (d, J=15.82 Hz, 1H), 6.96 (d, J=8.59 Hz, 1H), 7.18 (dd, J=8.40, 1.95 Hz, 1H), 7.39 (d, J=8.20 Hz, 1H), 7.43-7.47 (m, 1H), 7.44 (d, J=1.95 Hz, 1H), 7.52 (d, J=1.37 Hz, 1H).

### Intermediate 4: tert-butyl (2,3-dioxo-2,3-dihydro-1H-indol-1-yl)acetate

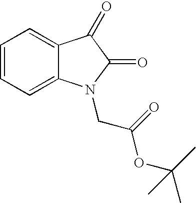

To a solution of isatin (100 mg, 0.68 mmol) in DMF (8.0 mL) was added potassium carbonate (235 mg, 1.70 mmol). The reaction was stirred at room temperature for 15 minutes. Tert-butyl bromoacetate (201 μL, 1.36 mmol) was then added. The reaction was stirred at room temperature overnight, concentrated in vacuo, dissolved in EtOAc and washed with H₂O (1×). The layers were separated and the aqueous layer was extracted with additional EtOAc (1×). The combined organic phases was dried over Na₂SO₄, filtered and concentrated in vacuo. The crude product was purified by silica gel column chromatography, eluting with 3:7 EtOAc:Hexanes, to give the title compound as an orange solid (167 mg, 94% yield). ¹H NMR (400 MHz, CDCl₃) δ 1.47 (s, 9H), 4.40 (s, 2H), 6.78 (d, J=8.01 Hz, 1H), 7.15 (dt, J=7.52, 0.78 Hz, 1H), 7.60 (dt, J=7.81, 1.37 Hz, 1H), 7.65 (ddd, J=7.62, 1.37, 0.59 Hz, 1H).

### Intermediate 5: tert-butyl (2,2′,5-trioxospiro[imidazolidine-4,3′-indol]-1′(2′H)yl)acetate

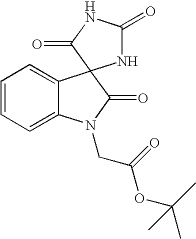

A mixture of tert-butyl (2,3-dioxo-2,3-dihydro-1H-indol-1-yl)acetate (750 mg, 2.87 mmol), potassium cyanide (224 mg, 3.44 mmol), and ammonium carbonate (2.65 g, 27.56 mmol) in 1:1 MeOH:H₂O (30 mL) was heated at 100° C. for 3 hours. The reaction was then cooled, concentrated in vacuo to remove the MeOH and filtered. The precipitate was rinsed with H₂O and EtOAc. The filtrate was diluted with H₂O and extracted with EtOAc (5×). The combined organic phases was dried over Na₂SO₄, filtered and concentrated in vacuo. The crude product was purified by silica gel column chromatography, eluting with 3:2. EtOAc:Hexanes, to give the title compound as a pale yellow solid (292 mg, 31% yield). ¹H NMR (400 MHz, CD₃OD) δ 1.46 (s, 9H), 4.47 (dd, J=30.46, 17.57 Hz, 2H), 6.99 (d, J=8.01 Hz, 1H), 7.17 (dt, J=7.62, 0.78 Hz, 1H), 7.34-7.37 (m, 1H), 7.42 (dt, J=7.81, 1.37 Hz, 1H).

### Intermediate 6: (2,2′,5-trioxospiro[imidazolidine-4,3′-indol]-1′(2′H)-yl)acetic acid

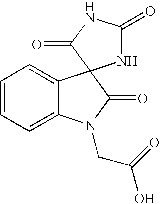

To a mixture of tert-butyl (2,2′,5-trioxospiro[imidazolidine-4,3′-indol]-1′(2′H)yl)acetate (292 mg, 0.88 mmol) in CH₂Cl₂ (3.5 mL) was added trifluoroacetic acid (3.5 mL). The reaction was stirred at room temperature for 90 minutes, concentrated in vacuo, azeotroped with toluene (2×) and left under vacuum overnight to be dried. Further purification of the residue was not necessary. The title compound was obtained as a yellow solid (242 mg, quantitative yield). ¹H NMR (400 MHz, CD₃OD) δ 4.54 (dd, J=54.48, 17.96 Hz, is 2H), 7.02 (d, J=8.01 Hz, 1H), 7.17 (dt, J=7.57, 0.88 Hz, 1H), 7.33-7.37 (m, J=7.42 Hz, 1H), 7.42 (dt, J=7.76, 1.27 Hz, 1H).

### Intermediate 9: [2-(3,4-dichlorophenyl)cyclopropyl]methanol

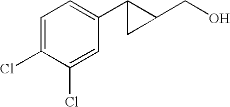

To a solution of diethylzinc (1.11 mL, 1.0M solution in hexanes) in CH₂Cl₂ (2 mL) at 0° C. was added diiodomethane (0.13 mL, 1.60 mmol). The reaction was stirred for 20 minutes during which a white precipitate formed. A solution of (2E)-3-(3,4-dichlorophenyl)prop-2-en-1-ol (100 mg, 0.49 mmol) in CH₂Cl₂ (1 mL) was then added to the reaction at 0° C. The reaction gradually warmed up to room temperature and was stirred overnight. The reaction was quenched with saturated NH₄Cl_{(aq)} and extracted with CH₂Cl₂ (3×). The combined organic phases was dried over Na₂SO₄, filtered and concentrated in vacuo. The crude product was purified by silica gel column chromatography, eluting with 3:2 EtOAc:Hexanes, to give the title compound as a colorless oil (77 mg, 72% yield). ¹H NMR (400 MHz, CDCl₃) δ 0.91-1.02 (m, 2H), 1.37-1.47 (m, 1H), 1.76-1.83 (m, 1H), 3.57-3.69 (m, 2H), 6.90 (dd, J=8.40, 2.15 Hz, 1H), 7.15 (d, J=2.15 Hz, 1H), 7.31 (d, J=8.40 Hz, 1H).

### Intermediate 10: 1,2-dichloro-4-[2-(chloromethyl)cyclopropyl]benzene

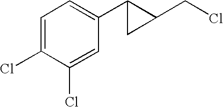

A solution of 1.5 M thionyl chloride (0.11 mL, 1.50 mmol) and benzotriazole (179 mg, 1.50 mmol) in CH₂Cl₂ (1 mL) was prepared. To a solution of [2-(3,4-dichlorophenyl)cyclopropyl]methanol (102 mg, 0.47 mmol) in CH₂Cl₂ (10 mL) was added the stock solution of thionyl chloride/benzotriazole (0.38 mL, 1.5 M). The reaction was stirred at room temperature for 30 minutes, filtered, and the precipitate was rinsed with CH₂Cl₂. The filtrate was washed with H₂O (1×) and 2% NaOH_{(aq)} (1×). The aqueous layers were extracted with additional CH₂Cl₂ (1×). The combined organic phases was dried over Na₂SO₄, filtered and concentrated in vacuo to give the title compound as a pale yellow oil with 90% purity (111 mg, 90% yield). Further purification of the product was not necessary. ¹H NMR (400 MHz, CDCl₃) δ 0.96-1.07 (m, 2H), 1.33-1.50 (m, 1H), 1.81-1.89 (m, 1H), 3.82-3.97 (m, 1H), 3.99-4.12 (m, 1H), 6.86-6.91 (m, 1H), 7.12-7.16 (m, 1H), 7.29-7.34 (m, 1H).

### Intermediate 11: 1-{[2-(3,4-dichlorophenyl)cyclopropyl]methyl}-1H-indole-2,3-dione

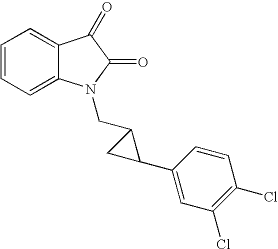

A solution of 1,2-dichloro-4-[2-(chloromethyl)cyclopropyl]benzene (51 mg, 0.22 mmol) and sodium iodide (81 mg, 0.54 mmol) in HPLC grade acetone was refluxed for 3 days. The reaction was concentrated in vacuo, diluted with ether, stirred for 45 minutes then filtered. The precipitate was rinsed with more ether. The filtrate was concentrated in vacuo to give a yellow oil. The residue was then dissolved in DMF (0.5 ml) and added to a mixture of isatin (29 mg, 0.20 mmol) and Cs₂CO₃ (71 mg, 0.22 mmol) in DMF (0.5 ml). The reaction was stirred at room temperature overnight, concentrated in vacuo, dissolved in EtOAc and washed with saturated NaHCO_{3(aq)} (1×). The aqueous layer was extracted with additional EtOAc (1×). The combined organic phases was dried over Na₂SO₄, filtered and concentrated in vacuo. The crude product was purified by silica gel column chromatography, eluting with 3:7 EtOAc:Hexanes, to give the title compound as a yellow solid is (29 mg, 42% yield). ¹H NMR (400 MHz, CDCl₃) δ 1.03 (dt, J=8.59, 5.47 Hz, 1H), 1.16 (dt, J=8.88, 5.52 Hz, 1H), 1.39-1.49 (m, 1H), 1.98-2.04 (m, 1H), 3.78 (dd, J=6.83 Hz, 2H), 6.85 (dd, J=8.30, 2.25 Hz, 1H), 6.98 (d, J=8.01 Hz, 1H), 7.06 (d, J=2.15 Hz, 1H), 7.14 (t, J=7.52 Hz, 1H), 7.28 (d, J=8.40 Hz, 1H), 7.58-7.65 (m, 2H).

### Intermediate 12:1-[(2E)-3-(3,4-dichlorophenyl)prop-2-en-1-yl]-1H-indole-2,3-dione

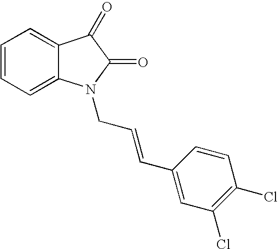

Using the same method as for 1-[(2E)-3-(3,4-dichlorophenyl)prop-2-en-1-yl]-5-(trifluoromethoxy)-1H-indole-2,3-dione and using isatin (200 mg, 1.36 mmol) and 1,2-dichloro-4-[(1E)-3-chloroprop-1-en-1-yl]benzene (150 mg, 0.36 mmol), except residue did not have to further purified after the work-up, afforded the title compound as an orange solid (341 mg, 76% yield). Purity (HPLC): >99%; ¹H NMR (400 MHz, CDCl₃) δ 4.53 (dd, J=5.86, 1.56 Hz, 2H), 6.20 (dt, J=15.82, 5.86 Hz, 1H), 6.57 (d, J=16.01 Hz, 1H), 6.92 (d, J=8.01 Hz, 1H), 7.12-7.20 (m, 2H), 7.38 (d, J=8.40 Hz, 1H), 7.43 (d, J=2.15 Hz, 1H), 7.58 (dt, J=7.81, 1.37 Hz, 1H), 7.65 (ddd, J=7.42, 1.37, 0.59 Hz, 1H). Found: C, 60.72; H, 3.40; N, 4.06. C₁₇H₁₁Cl₂NO₂+0.2H₂O has C, 60.81; H, 3.42; N, 4.17%.

### Intermediate 13: 1′-[(2E)-3-(3,4-dichlorophenyl)prop-2-en-1-yl]-2H,5H-spiro[imidazolidine-4,3′-indole]-2,2′,5(1′H)-trione

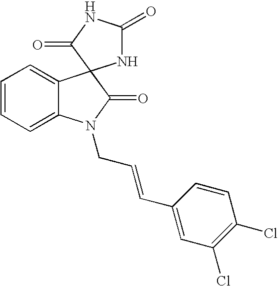

A mixture of 1-[(2E)-3-(3,4-dichlorophenyl)prop-2-en-1-yl]-1H-indole-2,3-dione (200 mg, 0.60 mmol), potassium cyanide (47 mg, 0.72 mmol), and ammonium carbonate (555 mg, 5.78 mmol) in 1:1 MeOH:H₂O (10 mL) was heated at 100° C. for 3 hours. The reaction was then cooled, concentrated in vacuo to remove the MeOH and filtered. The residue was dissolved in EtOAc and washed with H₂O (1×). The layers were separated and the aqueous layer was extracted with additional EtOAc (2×). The combined organic phases was dried over Na₂SO₄, filtered and concentrated in vacuo. The crude product was purified by silica gel column chromatography, eluting with 3:1 EtOAc:Hexanes, to give the title compound as a beige solid (189 mg, 78% yield). Purity (HPLC): >99%; ¹H NMR (400 MHz, CD₃OD) δ 4.49 (ddd, J=17.09, 5.08, 1.66 Hz, 1H), 4.61 (ddd, J=17.09, 4.98, 1.76 Hz, 1H), 6.35 (dt, J=16.01, 4.98 Hz, 1H), 6.58 (d, J=16.01 Hz, 1H), 7.08 (d, J=7.81 Hz, 1H), 7.17 (dt, J=7.62, 0.98 Hz, 1H), 7.30 (dd, J=8.59, 1.95 Hz, 1H), 7.35-7.38 (m, 1H), 7.39-7.44 (m, 2H), 7.52 (d, J=1.95 Hz, 1H). Found: C, 56.64; H, 3.26; N, 10.27. C₁₉H₁₃Cl₂N₃O₃ has C, 56.74; H, 3.26; N, 10.45%.

### Compound 3:1-[(2E)-3-(3,4-dichlorophenyl)prop-2-en-1-yl]-5-(trifluoromethoxy)-1H-indole-2,3-dione

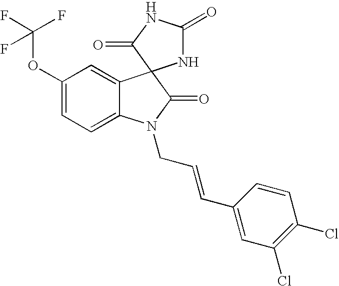

Using the same method as for 1′-[(2E)-3-(3,4-dichlorophenyl)prop-2-en-1-yl]-2H,5H-spiro[imidazolidine-4,3′-indole]-2,2′,5(1′H)-trione and using 1-[(2E)-3-(3,4-dichlorophenyl)prop-2-en-1-yl]-5-(trifluoromethoxy)-1H-indole-2,3-dione (150 mg, 0.36 mmol), except residue was purified by silica gel column chromatography, eluting with 1:1 EtOAc:Hexanes, afforded the title compound as a pale yellow solid (63 mg, 36% yield). Purity (HPLC): 94% (215 nm), >98% (254 nm); ¹H NMR (400 MHz, CD₃OD) δ 4.52 (ddd, J=17.14, 5.13, 1.76 Hz, 1H), 4.64 (ddd, J=16.99, 5.08, 1.76 Hz, 1H), 6.36 (dt, J=16.01, 5.08 Hz, 1H), 6.61 (d, J=16.01 Hz, 1H), 7.18 (d, J=8.59 Hz, 1H), 7.32 (dd, J=8.49, 1.85 Hz, 1H), 7.37 (ddd, J=8.59, 2.44, 0.88 Hz, 1H), 7.41-7.45 (m, 2H), 7.55 (d, J=1.95 Hz, 1H). Found: C, 50.39; H, 2.31; N, 8.35. C₂₀H₁₂Cl₂F₃N₃O₄+0.3 EtOAc has C, 50.14; H, 2.86; N, 8.27%.

### Compound 4: 1′-[(2E)-3-(3,4-dichlorophenyl)prop-2-en-1-yl]-1-methyl-2H,5H-spiro[imidazolidine-4,3′-indole]-2,2′,5(1′H)-trione

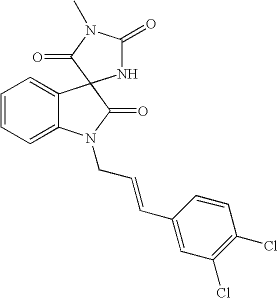

To a mixture of 1′-[(2E)-3-(3,4-dichlorophenyl)prop-2-en-1-yl]-2H,5H-spiro[imidazolidine-4,3′-indole]-2,2′,5(1′H)-trione (50 mg, 0.12 mmol) and potassium carbonate (34 mg, 0.25 mmol) in DMF (5 mL) was added iodomethane (19.3 μL, 0.31 mmol). The reaction was stirred at room temperature overnight and concentrated in vacuo to provide a mixture of two alkylated compounds. The residue was purified by reverse phase HPLC (gradient 40-70% CH₃CN in H₂O containing 0.1% trifluoroacetic acid) to give the title compound (25 mg, 49% yield) as its TFA salt. This material was lyophilized from CH₃CN/H₂O to produce a colorless solid. Purity (HPLC): >99%; ¹H NMR (400 MHz, CD₃OD) δ 3.07 (s, 3H), 4.50 (ddd, J=17.09, 5.13, 1.71 Hz, 1H), 4.61 (ddd, J=17.16, 5.00, 1.85 Hz, 1H), 6.35 (dt, J=−16.04, 5.06 Hz, 1H), 6.59 (dt, J=15.84, 1.50 Hz, 1H), 7:10 (d, J=7.91 Hz, 1H), 7.16 (dt, J=7.62, 0.98 Hz, 1H), 7.31 (dd, J=8.35, 2.10 Hz, 1H), 7.35 (ddd, J=7.49, 1.24, 0.54 Hz, 1H), 7.42 (dt, J=7.91, 1.27 Hz, 1H), 7.42 (d, J=8.40 Hz, 1H), 7.53 (d, J=2.05 Hz, 1H).

### Compound 5: 1′-[(2E)-3-(3,4-dichlorophenyl)prop-2-en-1-yl]-1,3-dimethyl-2H,5H-spiro[imidazolidine-4,3′-indole]-2,2′,5(1′H)-trione

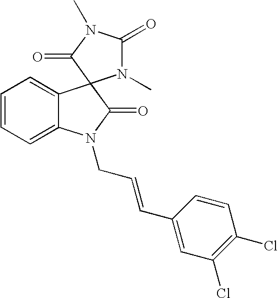

The second compound isolated from purification of the residue from the preparation of 1′-[(2E)-3-(3,4-dichlorophenyl)prop-2-en-1-yl]-1-methyl-2H,5H-spiro[imidazolidine-4,3′-indole]-2,2′,5(1′H)-trione was the TFA salt of the title compound (17 mg, 32%). This material was lyophilized from CH₃CN/H₂O to produce a beige solid. Purity (HPLC): >99%; ¹H NMR (400 MHz, CD₃OD) δ 2.75 (s, 3H), 3.09 (s, 3H), 4.48 (ddd, J=17.09, 5.17, 1.56 Hz, 1H), 4.68 (ddd, J=17.14, 4.93, 1.76 Hz, 1H), 6.37 (dt, J=16.06, 5.05 Hz, 1H), 6.60 (d, J=16.01 Hz, 1H), 7.15 (d, J=8.01 Hz, 1H), 7.19 (dt, J=7.62, 0.78 Hz, 1H), 7.31 (dd, J=8.40, 1.95 Hz, 1H), 7.34 (d, J=6.83 Hz, 1H), 7.41-7.44 (m, J=8.40 Hz, 1H), 7.47 (dt, J=7.81, 1.17 Hz, 1H), 7.54 (d, J=1.95 Hz, 1H).

### Compound 6: 1′-[(2E)-3-(3,4-dichlorophenyl)prop-2-en-1-yl]-1-(2-methoxyethyl)-2H,5H-spiro[imidazolidine-4,3′-indole]-2,2′,5(1′H)-trione

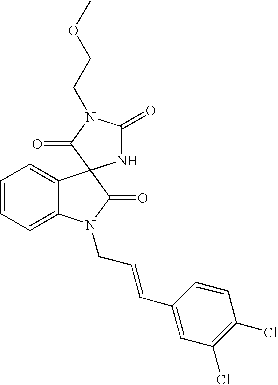

Using the same method as for 1′-[(2E)-3-(3,4-dichlorophenyl)prop-2-en-1-yl]-1-methyl-2H,5H-spiro[imidazolidine-4,3′-indole]-2,2′,5(1′H)-trione and using 1′-[(2E)-3-(3,4-dichlorophenyl)prop-2-en-1-yl]-2H,5H-spiro[imidazolidine-4,3′-indole]-2,2′,5(1′H)-trione (36 mg, 0.089 mmol), potassium carbonate (15 mg, 0.112 mmol) and 2-bromoethyl methyl ether (17 μL, 0.179 mmol) afforded a mixture of two alkylated compounds. The TFA salt of the title compound (10.1 mg, 25%) was obtained following purification of the residue by reverse phase HPLC (gradient 50-85% CH₃CN in H₂O containing 0.1% trifluoroacetic acid). This material was lyophilized from CH₃CN/H₂O to produce a pale yellow solid. Purity (HPLC): >99%; ¹H NMR (400 MHz, CD₃OD) δ 3.36 (s, 3H), 3.57-3.67 (m, 2H), 3.70-3.82 (m, 2H), 4.46-4.65 (m, 2H), 6.36 (dt, J=16.06, 5.05 Hz, 1H), 6.59 (d, J=16.21 Hz, 1H), 7.10 (d, J=7.81 Hz, 1H), 7.16 (dt, J=7.62, 0.98 Hz, 1H), 7.29-7.33 (m, 2H), 7.40-7.45 (m, 2H), 7.53 (d, J=1.95 Hz, 1H).

### Compound 7: 1′-[(2E)-3-(3,4-dichlorophenyl)prop-2-en-1-yl]-1,3-bis(2-methoxyethyl)-2H,5H-spiro[imidazolidine-4,3′-indole]-2,2′,5(1′H)-trione

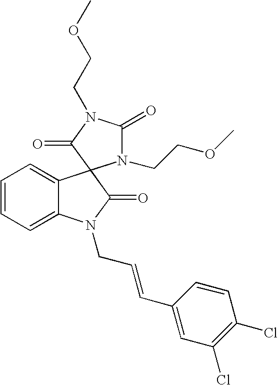

The second compound isolated from purification of the residue from the preparation of 1′-[(2E)-3-(3,4-dichlorophenyl)prop-2-en-1-yl]-1-(2-methoxyethyl)-2H,5H-spiro[imidazolidine-4,3′-indole]-2,2′,5(1′H)-trione above was the TFA salt of the title compound (9.9 mg, 21%). This material was lyophilized from CH₃CN/H₂O to produce a yellow hygroscopic solid. Purity (HPLC): 98% (215 nm), 96% (254 nm); ¹H NMR (400 MHz, CD₃OD) δ 2.92 (s, 3H), 3.26-3.38 (m, 2H), 3.34 (s, 3H), 3.56-3.84 (m, 6H), 4.45 (ddd, J 17.18, 5.08, 1.56 Hz, 1H), 4.68 (ddd, J=17.14, 4.83, 1.86 Hz, 1H), 6.37 (dt, J=16.16, 4.91 Hz, 1H), 6.65 (d, J=16.21 Hz, 1H), 7.10 (d, J=8.01 Hz, 1H), 7.16 (dt, J=7.52, 0.98 Hz, 1H), 7.24-7.28 (m, 1H), 7.31 (dd, J=8.40, 2.15 Hz, 1H), 7.41-7.46 (m, 2H), 7.53 (d, J=2.15 Hz, 1H).

### Compound 8: N-phenyl-2-(2,2′,5-trioxospiro[imidazolidine-4,3′-indol]-1′(2′H)yl)acetamide

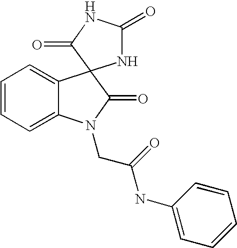

A suspension of (2,2′,5-trioxospiro[imidazolidine-4,3′-indol]-1′(2′H)-yl)acetic acid (100 mg, 0.36 mmol) in thionyl chloride (1 mL) was heated at 80° C. for 24 hours, concentrated in vacuo, azeotroped with toluene (2×), and placed under vacuum overnight to be dried. The residue was suspended in CH₂Cl₂ (3.2 mL). DMA (0.32 mL) was added, followed by aniline (36.5 μL, 0.40 mmol) and triethylamine (76 μL, 0.55 mmol). The reaction was stirred at room temperature overnight, concentrated in vacuo, dissolved in EtOAc and washed with saturated NaHCO_{3(aq)} (1×) and brine (1×). The aqueous layers were extracted with additional EtOAc (2×). The combined organic phases was dried over Na₂SO₄, filtered and concentrated in vacuo. The residue was purified by reverse phase HPLC (gradient 30-60% CH₃CN in H₂O containing 0.1% trifluoroacetic acid) to give the title compound (66 mg, 52% yield) as its TFA salt. This material was lyophilized from CH₃CN/H₂O to produce a beige solid. Purity (HPLC): >99%; ¹H NMR (400 MHz, CD₃OD) δ 4.54 (d, J=16.99 Hz, 1H), 4.73 (d, J=16.79 Hz, 1H), 7.06 (d, J=7.81 Hz, 1H), 7.10 (t, J=7.42 Hz, 1H), 7.18 (dt, J=7.57, 0.88 Hz, 1H), 7.30 (t, J=8.01 Hz, 2H), 7.37 (d, J=7.42 Hz, 1H), 7.42 (dt, J=7.81, 1.17 Hz, 1H), 7.54 (dd, J=8.69, 1.07 Hz, 2H). Found: C, 59.03; H, 4.04; N, 14.84. C₁₈H₁₄N₄O₄+0.1H₂O+0.2 TFA has C, 58.94; H, 3.87; N, 14.94%.

### Compound 9: N-(3,4-difluorophenyl)-2-(2,2′,5-trioxospiro[imidazolidine-4,3′-indol]-1′(2′H)-yl)acetamide

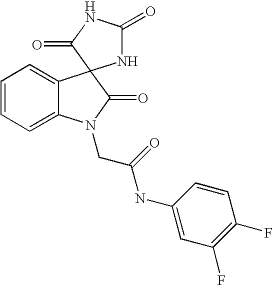

Using the same method as for N-phenyl-2-(2,2′,5-trioxospiro[imidazolidine-4,3′-indol]-1′(2′H)-yl)acetamide and using (2,2′,5-trioxospiro[imidazolidine-4,3′-indol]-1′(2′H)yl)acetic acid (80 mg, 0.29 mmol), and 3,4-difluoroaniline (32 μL, 0.32 mmol) afforded the title compound (72 mg, 64%) as its TFA salt. This material was lyophilized from CH₃CN/H₂O to produce a beige solid. Purity (HPLC): >99%; ¹H NMR (400 MHz, CD₃OD) δ 4.54 (d, J=16.99 Hz, 1H), 4.71 (d, J=16.99 Hz, 1H), 7.05 (d, J=7.81 Hz, 1H), 7.16-7.26 (m, 3H), 7.36-7.39 (m, 1H), 7.42 (dt, J=7.81, 1.37 Hz, 1H), 7.64-7.69 (m, 1H). Found: C, 56.42; H, 3.37; N, 14.42. C₁₈H₁₂F₂N₄O₄ has C, 55.96; H, 3.13; N, 14.50

### Compound 10: N-pyridin-3-yl-2-(2,2′,5-trioxospiro[imidazolidine-4,3′-indol]-1′(2′H)yl)acetamide

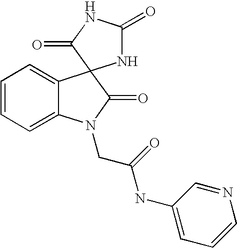

A suspension of (2,2′,5-trioxospiro[imidazolidine-4,3′-indol]-1′(2′H)-yl)acetic acid (88 mg, 0.32 mmol) in thionyl chloride (1 mL) was heated at 80° C. for 24 hours, concentrated in vacuo, azeotroped with toluene (2×), and placed under vacuum overnight to be dried. The residue was suspended in CH₂Cl₂ (3.5 mL). DMA (0.35 mL) was added, followed by 3-aminopyridine (33 mg, 0.35 mmol) and triethylamine (67 μL, 0.48 mmol). The reaction was stirred at room temperature overnight and concentrated in vacuo. The residue was purified by reverse phase HPLC (gradient 1-20% CH₃CN in H₂O containing 0.1% trifluoroacetic acid) to give the title compound (58 mg, 51% yield) as its TFA salt. This material was lyophilized from CH₃CN/H₂O to produce a pale yellow solid. Purity (HPLC): 98%; ¹H NMR (400 MHz, CD₃OD) δ 4.64-4.81 (m, 2H), 7.08 (d, J=7.81 Hz, 1H), 7.20 (dd, J=7.62, 0.78 Hz, 1H), 7.39 (dd, J=7.52, 0.68 Hz, 1H), 7.84 (dd, J=8.59, 5.47 Hz, 1H), 8.34-8.39 (m, 1H), 8.48 (d, J=4.49 Hz, 1H), 9.16 (s, 1H). Found: C, 46.55; H, 2.68; N, 13.10. C₁₇H₁₃N₅O₄+0.1H₂O+1.4 TFA has C, 46.38; H, 2.87; N, 13.66%.

### Compound 11: N-(4-methoxyphenyl)-2-(2,2′,5-trioxospiro[imidazolidine-4,3′-indol]-1′(2′H)-yl)acetamide

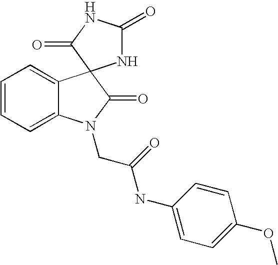

Using the same method as for N-phenyl-2-(2,2′,5-trioxospiro[imidazolidine-4,3′-indol]-1′(2′H)-yl)acetamide and using (2,2′,5-trioxospiro[imidazolidine-4,3′-indol]-1′(2′H)yl)acetic acid (80 mg, 0.29 mmol), and 4-methoxyaniline (39 mg, 0.32 mmol), except the residue was purified by reverse phase HPLC (gradient 15-40% CH₃CN in H₂O containing 0.1% trifluoroacetic acid), afforded the title compound (40 mg, 36%) as its TFA salt. This material was lyophilized from CH₃CN/H₂O to produce a beige solid. Purity (HPLC): >99%; ¹H NMR (400 MHz, CD₃OD) δ 3.76 (s, 3H), 4.51 (d, J=16.79 Hz, 1H), 4.71 (d, J=16.79 Hz, 1H), 6.84-6.89 (m, 2H), 7.05 (d, J=7.81 Hz, 1H), 7.18 (dt, J=7.57, 0.88 Hz, 1H), 7.37 (d, J=6.83 Hz, 1H), 7.40-7.46 (m, 3H). Found: C, 57.49; H, 3.94; N, 13.24. C₁₉H₁₆N₄O₅+0.2H₂O+0.2 TFA has C, 57.28; H, 4.11; N, 13.77.

### Compound 12: N-(3,4-dichlorophenyl)-2-(2,2′,5-trioxospiro[imidazolidine-4,3′-indol]-1′(2′H)-yl)acetamide

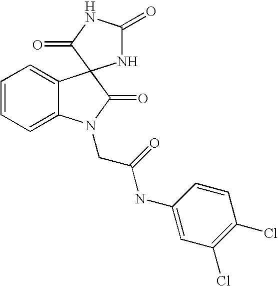

Using the same method as for N-phenyl-2-(2,2′,5-trioxospiro[imidazolidine-4,3′-indol]-1′(2′H)-yl)acetamide and using (2,2′,5-trioxospiro[imidazolidine-4,3′-indol]-1′(2′H)yl)acetic acid (80 mg, 0.29 mmol), and 3,4-dichloroaniline (52 mg, 0.32 mmol), except the residue was purified by reverse phase HPLC (gradient 25-50% CH₃CN in H₂O containing 0.1% trifluoroacetic acid), afforded the title compound (50 mg, 41%) as its TFA salt. This material was lyophilized from CH₃CN/H₂O to produce a beige solid. Purity (HPLC): >99%; ¹H NMR (400 MHz, CD₃OD) δ 3.76 (s, 3H), 4.51 (d, J=16.79 Hz, 1H), 4.71 (d, J=16.79 Hz, 1H), 6.84-6.89 (m, 2H), 7.05 (d, J=7.81 Hz, 1H), 7.18 (dt, J=7.57, 0.88 Hz, 1H), 7.37 (d, J=6.83 Hz, 1H), 7.40-7.46 (m, 3H). Found: C, 51.57; H, 2.76; N, 12.57. C₁₈H₁₂Cl₂N₄O₄+0.1H₂O has C, 51.35; H, 2.92; N, 13.31%.

### Compound 14: 1′-{[2-(3,4-dichlorophenyl)cyclopropyl]methyl}-2H,5H-spiro[imidazolidine-4,3′-indole]-2,2′,5(1′H)-trione

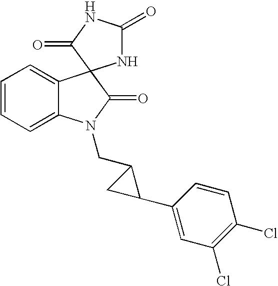

A mixture of 1-{[2-(3,4-dichlorophenyl)cyclopropyl]methyl}-1H-indole-2,3-dione (29 mg, 0.084 mmol), potassium cyanide (6.5 mg, 0.101 mmol), and ammonium carbonate (77 mg, 0.804 mmol) in 1:1 MeOH:H₂O (2 mL) was heated at 100° C. for 3 hours. The reaction was then cooled and concentrated in vacuo. The residue was purified by reverse phase HPLC (gradient 50-80% CH₃CN in H₂O containing 0.1% trifluoroacetic acid) to give the title compound (7.5 mg, 21% yield) as its TFA salt. This material was lyophilized from CH₁₃CN/H₂O to produce a pale yellow solid. Purity (HPLC): 94% (215 nm), 94% (254 nm); ¹H NMR (400 MHz, CD₃OD) δ 0.98-1.16 (m, 2H), 1.43-1.58 (m, 1H), 1.99-2.07 (m, 1H), 3.76-3.85 (m, 1H), 3.86-3.93 (m, 1H), 6.96 (dt, J=8.35, 1.59 Hz, 1H), 7.16 (t, J=7.52 Hz, 1H), 7.19-7.23 (m, 2H), 7.28-7.35 (m, 2H), 7.44 (tt, J=7.81, 1.46 Hz, 1H).

### General Procedure 1:

As illustrated in Scheme 4, stock solutions (0.375 M) of the alkyl halides (187.5 mmol/well) in DMF (500 μL/well) were prepared. Stock solutions (0.25 M) of the isatins (125 μmol/well) in DMF (500 μL/well) were also prepared. PS-TBD (˜130 mg/well, 1.48 mmol/g) was dispensed into Robbins blocks equipped with filters followed by the isatin stock solutions (500 μL/well) and DMF (500 μL/well). The reactions were mixed for 1 is hour at room temperature. The alkyl halide stock solutions (500 μL/well) were then added and the reactions were heated at 50° C. for 4 days, and then filtered into a 96-well plate. The Robbins blocks were rinsed with DMF. The filtrates were combined and concentrated in vacuo. The crude alkylated isatins were transferred to Robbins blocks equipped with filters using DMA (500 μL/well). Ammonium carbonate (˜130 mg/well) was dispensed into the Robbins block, followed by H₂O (400 μL/well) and a solution of KCN in H₂O (100 μL/well, 3.75 M). The reactions were heated at 50° C. for 24 hours, and then filtered into a 96-well plate. The Robbins blocks were rinsed with DMA. The filtrates were combined and concentrated in vacuo. The residues were dissolved in EtOAc (700 μL/well) and washed with H₂O (500 μL/well). The organic layer was transferred into a new plate. The aqueous layer was extracted with more EtOAc (3×700 μL/well). The organic layers were combined and concentrated in vacuo. The products were purified by reverse phase HPLC to provide the corresponding hydantoins.

| | | Retention | |
|---|---|---|---|
| Example. # | Name (IUPAC) | Time | MH+ |
| 15 | 1′-(2-methylpropyl)-2H,5H-spiro[imidazolidine-4,3′-indole]- | 1.16 | 273.48 |
| | 2,2′,5(1′H)-trione | | |
| 16 | 1′-(2-ethylbutyl)-2H,5H-spiro[imidazolidine-4,3′-indole]- | 1.41 | 301.45 |
| | 2,2′,5(1′H)-trione | | |
| 17 | 1′-[(2,6-dichloro-4-pyridinyl)methyl]-2H,5H- | 1.37 | 376.15 |
| | spiro[imidazolidine-4,3′-indole]-2,2′,5(1′H)-trione | | |
| 18 | 1′-{[4-(methylsulfonyl)phenyl]methyl}-2H,5H- | 1.13 | 385.2 |
| | spiro[imidazolidine-4,3′-indole]-2,2′,5(1′H)-trione | | |
| 19 | 1′-[2-(1H-indol-3-yl)ethyl]-2H,5H-spiro[imidazolidine-4,3′- | 1.41 | 360.3 |
| | indole]-2,2′,5(1′H)-trione | | |
| 20 | 1′-[3-(methyloxy)propyl]-2H,5H-spiro[imidazolidine-4,3′- | 0.88 | 289.44 |
| | indole]-2,2′,5(1′H)-trione | | |
| 21 | 1′-(cyclopropylmethyl)-2H,5H-spiro[imidazolidine-4,3′- | 1.08 | 271.47 |
| | indole]-2,2′,5(1′H)-trione | | |
| 22 | 1′-(2-{[2-(methyloxy)phenyl]oxy}ethyl)-2H,5H- | 1.35 | 367.27 |
| | spiro[imidazolidine-4,3′-indole]-2,2′,5(1′H)-trione | | |
| 23 | 1′-[(2-chloro-1,3-thiazol-5-yl)methyl]-2H,5H- | 1.15 | 348.21 |
| | spiro[imidazolidine-4,3′-indole]-2,2′,5(1′H)-trione | | |
| 24 | 1′-{[5-(4-chlorophenyl)-1,3,4-oxadiazol-2-yl]methyl}- | 1.41 | 409.12 |
| | 2H,5H-spiro[imidazolidine-4,3′-indole]-2,2′,5(1′H)-trione | | |
| 25 | 1′-(cyclohexylmethyl)-2H,5H-spiro[imidazolidine-4,3′- | 1.43 | 313.42 |
| | indole]-2,2′,5(1′H)-trione | | |
| 26 | 1′-(phenylmethyl)-2H,5H-spiro[imidazolidine-4,3′-indole]- | 1.32 | 307.39 |
| | 2,2′,5(1′H)-trione | | |
| 27 | 1′-[2-(methyloxy)ethyl]-2H,5H-spiro[imidazolidine-4,3′- | 0.73 | 275.46 |
| | indole]-2,2′,5(1′H)-trione | | |
| 28 | 5′-chloro-1′-(2-phenylethyl)-2H,5H-spiro[imidazolidine-4,3′- | 1.48 | 355.26 |
| | indole]-2,2′,5(1′H)-trione | | |
| 29 | 5′-chloro-1′-(2-{[2-(methyloxy)phenyl]oxy}ethyl)-2H,5H- | 1.48 | 401.15 |
| | spiro[imidazolidine-4,3′-indole]-2,2′,5(1′H)-trione | | |
| 30 | 5′-chloro-1′-(3-methylbutyl)-2H,5H-spiro[imidazolidine-4,3′- | 1.45 | 321.36 |
| | ndole]-2,2′,5(1′H)-trione | | |
| 31 | 5′-chloro-1′-[(6-chloro-3-pyridinyl)methyl]-2H,5H- | 1.33 | 376.17 |
| | spiro[imidazolidine-4,3′-indole]-2,2′,5(1′H)-trione | | |
| 32 | 1′-[(2E)-2-butenyl]-5′-chloro-2H,5H-spiro[imidazolidine- | 1.33 | 305.37 |
| | 4,3′-indole]-2,2′,5(1′H)-trione | | |
| 33 | 1′-[(2-bromophenyl)methyl]-5′-fluoro-2H,5H- | 1.57 | 403.04 |
| | spiro[imidazolidine-4,3′-indole]-2,2′,5(1′H)-trione | | |
| 34 | 5′-fluoro-1′-(2-methylpropyl)-2H,5H-spiro[imidazolidine- | 1.22 | 291.41 |
| | 4,3′-indole]-2,2′,5(1′H)-trione | | |
| 35 | 5′-fluoro-1′-{[4-(1-methylethyl)phenyl]methyl}-2H,5H- | 1.6 | 367.26 |
| | spiro[imidazolidine-4,3′-indole]-2,2′,5(1′H)-trione | | |
| 36 | 5′-fluoro-1′-{[3-(methyloxy)phenyl]methyl}-2H,5H- | 1.38 | 355.26 |
| | spiro[imidazolidine-4,3′-indole]-2,2′,5(1′H)-trione | | |
| 37 | 5′-fluoro-1′-(2-phenylethyl)-2H,5H-spiro[imidazolidine-4,3′- | 1.38 | 339.31 |
| | indole]-2,2′,5(1′H)-trione | | |
| 38 | 5′-fluoro-1′-(4-methyl-3-pentenyl)-2H,5H- | 1.38 | 317.37 |
| | spiro[imidazolidine-4,3′-indole]-2,2′,5(1′H)-trione | | |
| 39 | 5′-fluoro-1′-{[2-(trifluoromethyl)phenyl]methyl}-2H,5H- | 1.52 | 393.16 |
| | spiro[imidazolidine-4,3′-indole]-2,2′,5(1′H)-trione | | |
| 40 | 1′-(2-ethylbutyl)-5′-fluoro-2H,5H-spiro[imidazolidine-4,3′- | 1.43 | 319.39 |
| | indole]-2,2′,5(1′H)-trione | | |
| 41 | 5′-fluoro-1′-{2-[(4-fluorophenyl)oxy]ethyl}-2H,5H- | 1.45 | 373.22 |
| | spiro[imidazolidine-4,3′-indole]-2,2′,5(1′H)-trione | | |
| 42 | 1′-[(2,6-dichloro-4-pyridinyl)methyl]-5′-fluoro-2H,5H- | 1.4 | 394.09 |
| | spiro[imidazolidine-4,3′-indole]-2,2′,5(1′H)-trione | | |
| 43 | 1′-(1,1′-biphenyl-2-ylmethyl)-5′-fluoro-2H,5H- | 1.65 | 401.18 |
| | spiro[imidazolidine-4,3′-indole]-2,2′,5(1′H)-trione | | |
| 44 | 5′-fluoro-1′-{[4-(methylsulfonyl)phenyl]methyl}-2H,5H- | 1.2 | 403.13 |
| | spiro[imidazolidine-4,3′-indole]-2,2′,5(1′H)-trione | | |
| 45 | 5′-fluoro-1′-[2-(1H-indol-3-yl)ethyl]-2H,5H- | 1.45 | 378.24 |
| | spiro[imidazolidine-4,3′-indole]-2,2′,5(1′H)-trione | | |
| 46 | 1′-{[2-chloro-5-(trifluoromethyl)phenyl]methyl}-5′-fluoro- | 1.6 | 427.05 |
| | 2H,5H-spiro[imidazolidine-4,3′-indole]-2,2′,5(1′H)-trione | | |
| 47 | 5′-fluoro-1′-{[6-(trifluoromethyl)-3-pyridinyl]methyl}- | 1.35 | 394.14 |
| | 2H,5H-spiro[imidazolidine-4,3′-indole]-2,2′,5(1′H)-trione | | |
| 48 | 5′-fluoro-1′-[3-(methyloxy)propyl]-2H,5H- | 0.95 | 307.39 |
| | spiro[imidazolidine-4,3′-indole]-2,2′,5(1′H)-trione | | |
| 49 | 5′-fluoro-1′-{[5-(trifluoromethyl)-2-furanyl]methyl}-2H,5H- | 1.45 | 383.17 |
| | spiro[imidazolidine-4,3′-indole]-2,2′,5(1′H)-trione | | |
| 50 | 5′-fluoro-1′-[(2,4,6-trimethylphenyl)methyl]-2H,5H- | 1.58 | 367.29 |
| | spiro[imidazolidine-4,3′-indole]-2,2′,5(1′H)-trione | | |
| 51 | 1′-[(2-chloro-6-fluorophenyl)methyl]-5′-fluoro-2H,5H- | 1.41 | 377.17 |
| | spiro[imidazolidine-4,3′-indole]-2,2′,5(1′H)-trione | | |
| 52 | 1′-(cyclopropylmethyl)-5′-fluoro-2H,5H-spiro[imidazolidine- | 1.15 | 289.42 |
| | 4,3′-indole]-2,2′,5(1′H)-trione | | |
| 53 | 5′-fluoro-1′-[2-(phenyloxy)ethyl]-2H,5H-spiro[imidazolidine- | 1.41 | 355.28 |
| | 4,3′-indole]-2,2′,5(1′H)-trione | | |
| 54 | 5′-fluoro-1′-[2-(1H-pyrrol-l-yl)ethyl]-2H,5H- | 1.23 | 328.34 |
| | spiro[imidazolidine-4,3′-indole]-2,2′,5(1′H)-trione | | |
| 55 | 5′-fluoro-1′-[(5-methyl-3-isoxazolyl)methyl]-2H,5H- | 1.13 | 330.32 |
| | spiro[imidazolidine-4,3′-indole]-2,2′,5(1′H)-trione | | |
| 56 | 5′-fluoro-1′-(2-{[2-(methyloxy)phenyl]oxy}ethyl)-2H,5H- | 1.4 | 385.22 |
| | spiro[imidazolidine-4,3′-indole]-2,2′,5(1′H)-trione | | |
| 57 | 1′-[(2-chloro-1,3-thiazol-5-yl)methyl]-5′-fluoro-2H,5H- | 1.22 | 366.16 |
| | spiro[imidazolidine-4,3′-indole]-2,2′,5(1′H)-trione | | |
| 58 | 1′-[(2,3-dichlorophenyl)methyl]-5′-fluoro-2H,5H- | 1.58 | 393.11 |
| | spiro[imidazolidine-4,3′-indole]-2,2′,5(1′H)-trione | | |
| 59 | 5′-fluoro-1′-({5-[4-(methyloxy)phenyl]-1,3,4-oxadiazol-2- | 1.37 | 423.12 |
| | yl}methyl)-2H,5H-spiro[imidazolidine-4,3′-indole]- | | |
| | 2,2′,5(1′H)-trione | | |
| 60 | 5′-fluoro-1′-(3-methylbutyl)-2H,5H-spiro[imidazolidine-4,3′- | 1.37 | 305.41 |
| | indole]-2,2′,5(1′H)-trione | | |
| 61 | 1′-(cyclohexylmethyl)-5′-fluoro-2H,5H-spiro[imidazolidine- | 1.48 | 331.37 |
| | 4,3′-indole]-2,2′,5(1′H)-trione | | |
| 62 | 1′-[(6-chloro-3-pyridinyl)methyl]-5′-fluoro-2H,5H- | 1.25 | 360.21 |
| | spiro[imidazolidine-4,3′-indole]-2,2′,5(1′H)-trione | | |
| 63 | 5′-fluoro-1′-(2-propynyl)-2H,5H-spiro[imidazolidine-4,3′- | 1.01 | 273.44 |
| | indole]-2,2′,5(1′H)-trione | | |
| 64 | 5′-fluoro-1′-pentyl-2H,5H-spiro[imidazolidine-4,3′-indole]- | 1.37 | 305.42 |
| | 2,2′,5(1′H)-trione | | |
| 65 | 1′-(cyclobutylmethyl)-5′-fluoro-2H,5H-spiro[imidazolidine- | 1.28 | 303.41 |
| | 4,3′-indole]-2,2′,5(1′H)-trione | | |
| 66 | 1′-[2-(ethyloxy)ethyl]-5′-fluoro-2H,5H-spiro[imidazolidine- | 1.03 | 307.4 |
| | 4,3′-indole]-2,2′,5(1′H)-trione | | |
| 67 | 5′-fluoro-1′-{[4-(1,2,3-thiadiazol-4-yl)phenyl]methyl}- | 1.41 | 409.13 |
| | 2H,5H-spiro[imidazolidine-4,3′-indole]-2,2′,5(1′H)-trione | | |
| 68 | 5′-fluoro-1′-(phenylmethyl)-2H,5H-spiro[imidazolidine-4,3′- | 1.37 | 325.35 |
| | indole]-2,2′,5(1′H)-trione | | |
| 69 | 1′-[(2E)-2-butenyl]-5′-fluoro-2H,5H-spiro[imidazolidine-4,3′- | 1.22 | 289.43 |
| | indole]-2,2′,5(1′H)-trione | | |
| 70 | 5′-fluoro-1′-[2-(methyloxy)ethyl]-2H,5H-spiro[imidazolidine- | 0.88 | 293.42 |
| | 4,3′-indole]-2,2′,5(1′H)-trione | | |
| 71 | 5′-fluoro-1′-(2-propenyl)-2H,5H-spiro[imidazolidine-4,3′- | 1.05 | 275.45 |
| | indole]-2,2′,5(1′H)-trione | | |
| 72 | 5′-fluoro-1′-[(2E)-3-phenyl-2-propenyl]-2H,5H- | 1.5 | 351.3 |
| | spiro[imidazolidine-4,3′-indole]-2,2′,5(1′H)-trione | | |
| 73 | 1′-[(3,5-dimethyl-4-isoxazolyl)methyl]-5′-fluoro-2H,5H- | 1.15 | 344.3 |
| | spiro[imidazolidine-4,3′-indole]-2,2′,5(1′H)-trione | | |
| 74 | 5′-chloro-7′-methyl-1′-{[3-(methyloxy)phenyl]methyl}- | 1.55 | 385.19 |
| | 2H,5H-spiro[imidazolidine-4,3′-indole]-2,2′,5(1′H)-trione | | |
| 75 | 5′-chloro-1′-{2-[(4-fluorophenyl)oxy]ethyl}-7′-methyl- | 1.6 | 403.14 |
| | 2H,5H-spiro[imidazolidine-4,3′-indole]-2,2′,5(1′H)-trione | | |
| 76 | 5′-chloro-1′-{[2-fluoro-4-(trifluoromethyl)phenyl]methyl}-7′- | 1.7 | 441.03 |
| | methyl-2H,5H-spiro[imidazolidine-4,3′-indole]-2,2′,5(1′H)- | | |
| | trione | | |
| 77 | 5′-chloro-1′-{[4-fluoro-3-(trifluoromethyl)phenyl]methyl}-7′- | 1.66 | 441.03 |
| | methyl-2H,5H-spiro[imidazolidine-4,3′-indole]-2,2′,5(1′H)- | | |
| | trione | | |
| 78 | 5′-chloro-1′-[(2-chloro-6-fluorophenyl)methyl]-7′-methyl- | 1.58 | 407.08 |
| | 2H,5H-spiro[imidazolidine-4,3′-indole]-2,2′,5(1′H)-trione | | |
| 79 | 5′-chloro-7′-methyl-1′-{[3-(trifluoromethyl)phenyl]methyl}- | 1.65 | 423.09 |
| | 2H,5H-spiro[imidazolidine-4,3′-indole]-2,2′,5(1′H)-trione | | |
| 80 | 5′-chloro-7′-methyl-1′-{[4-(trifluoromethyl)phenyl]methyl}- | 1.66 | 423.09 |
| | 2H,5H-spiro[imidazolidine-4,3′-indole]-2,2′,5(1′H)-trione | | |
| 81 | 5′-chloro-7′-methyl-1′-[2-(1H-pyrrol-1-yl)ethyl]-2H,5H- | 1.45 | 358.26 |
| | spiro[imidazolidine-4,3′-indole]-2,2′,5(1′H)-trione | | |
| 82 | 5′-chloro-1′-(imidazo[1,2-a]pyridin-2-ylmethyl)-7′-methyl- | 1.06 | 395.17 |
| | 2H,5H-spiro[imidazolidine-4,3′-indole]-2,2′,5(1′H)-trione | | |
| 83 | 5′-chloro-7′-methyl-1′-(3-methylbutyl)-2H,5H- | 1.53 | 335.32 |
| | spiro[imidazolidine-4,3′-indole]-2,2′,5(1′H)-trione | | |
| 84 | 5′-chloro-1′-(cyclohexylmethyl)-7′-methyl-2H,5H- | 1.63 | 361.28 |
| | spiro[imidazolidine-4,3′-indole]-2,2′,5(1′H)-trione | | |
| 85 | 5′-chloro-1′-[(6-chloro-3-pyridinyl)methyl]-7′-methyl-2H,5H- | 1.41 | 390.12 |
| | spiro[imidazolidine-4,3′-indole]-2,2′,5(1′H)-trione | | |
| 86 | 5′-chloro-7′-methyl-1′-pentyl-2H,5H-spiro[imidazolidine- | 1.55 | 335.33 |
| | 4,3′-indole]-2,2′,5(1′H)-trione | | |
| 87 | 5′-chloro-1′-(cyclobutylmethyl)-7′-methyl-2H,5H- | 1.48 | 333.32 |
| | spiro[imidazolidine-4,3′-indole]-2,2′,5(1′H)-trione | | |
| 88 | 5′-chloro-1′-[2-(ethyloxy)ethyl]-7′-methyl-2H,5H- | 1.32 | 337.3 |
| | spiro[imidazolidine-4,3′-indole]-2,2′,5(1′H)-trione | | |
| 89 | 5′-chloro-7′-methyl-1′-(phenylmethyl)-2H,5H- | 1.53 | 355.25 |
| | spiro[imidazolidine-4,3′-indole]-2,2′,5(1′H)-trione | | |
| 90 | 1′-[(2E)-2-butenyl]-5′-chloro-7′-methyl-2H,5H- | 1.43 | 319.33 |
| | spiro[imidazolidine-4,3′-indole]-2,2′,5(1′H)-trione | | |
| 91 | 5′-chloro-7′-methyl-1′-[2-(methyloxy)ethyl]-2H,5H- | 1.23 | 323.32 |
| | spiro[imidazolidine-4,3′-indole]-2,2′,5(1′H)-trione | | |
| 92 | 5′-chloro-7′-methyl-1′-(2-propenyl)-2H,5H- | 1.32 | 305.35 |
| | spiro[imidazolidine-4,3′-indole]-2,2′,5(1′H)-trione | | |
| 93 | 5′-chloro-1′-[(3,5-dimethyl-4-isoxazolyl)methyl]-7′-methyl- | 1.35 | 374.21 |
| | 2H,5H-spiro[imidazolidine-4,3′-indole]-2,2′,5(1′H)-trione | | |
| 94 | 5′-methyl-1′-(2-methylpropyl)-2H,5H-spiro[imidazolidine- | 1.3 | 287.46 |
| | 4,3′-indole]-2,2′,5(1′H)-trione | | |
| 95 | 1′-(2,1,3-benzoxadiazol-5-ylmethyl)-2H,5H- | 1.32 | 349.29 |
| | spiro[imidazolidine-4,3′-indole]-2,2′,5(1′H)-trione | | |
| 96 | 1′-[(2-chloro-6-fluorophenyl)methyl]-2H,5H- | 1.38 | 359.22 |
| | spiro[imidazolidine-4,3′-indole]-2,2′,5(1′H)-trione | | |
| 97 | 1′-[2-(1H-pyrrol-1-yl)ethyl]-2H,5H-spiro[imidazolidine-4,3′- | 1.15 | 310.39 |
| | indole]-2,2′,5(1′H)-trione | | |
| 98 | 1′-(3-methylbutyl)-2H,5H-spiro[imidazolidine-4,3′-indole]- | 1.32 | 287.46 |
| | 2,2′,5(1′H)-trione | | |
| 99 | 1′-[(6-chloro-3-pyridinyl)methyl]-2H,5H- | 1.16 | 342.26 |
| | spiro[imidazolidine-4,3′-indole]-2,2′,5(1′H)-trione | | |
| 100 | 1′-pentyl-2H,5H-spiro[imidazolidine-4,3′-indole]-2,2′,5(1′H)- | 1.33 | 287.45 |
| | trione | | |
| 101 | 1′-(cyclobutylmethyl)-2H,5H-spiro[imidazolidine-4,3′- | 1.23 | 285.45 |
| | indole]-2,2′,5(1′H)-trione | | |
| 102 | 1′-[2-(ethyloxy)ethyl]-2H,5H-spiro[imidazolidine-4,3′- | 0.92 | 289.44 |
| | indole]-2,2′,5(1′H)-trione | | |
| 103 | 1′-[(2E)-2-butenyl]-2H,5H-spiro[imidazolidine-4,3′-indole]- | 1.15 | 271.47 |
| | 2,2′,5(1′H)-trione | | |
| 104 | 1′-(2-propenyl)-2H,5H-spiro[imidazolidine-4,3′-indole]- | 0.97 | 257.49 |
| | 2,2′,5(1′H)-trione | | |
| 105 | 1′-[(3,5-dimethyl-4-isoxazolyl)methyl]-2H,5H- | 1.08 | 326.34 |
| | spiro[imidazolidine-4,3′-indole]-2,2′,5(1′H)-trione | | |
| 106 | 1′-[(4-fluorophenyl)methyl]-5′-methyl-2H,5H- | 1.43 | 339.31 |
| | spiro[imidazolidine-4,3′-indole]-2,2′,5(1′H)-trione | | |
| 107 | 1′-[(2-bromophenyl)methyl]-5′-methyl-2H,5H- | 1.53 | 399.1 |
| | spiro[imidazolidine-4,3′-indole]-2,2′,5(1′H)-trione | | |
| 108 | 5′-methyl-1′-{[4-(1-methylethyl)phenyl]methyl}-2H,5H- | 1.63 | 363.32 |
| | spiro[imidazolidine-4,3′-indole]-2,2′,5(1′H)-trione | | |
| 109 | 5′-methyl-1′-{[3-(methyloxy)phenyl]methyl}-2H,5H- | 1.43 | 352.33 |
| | spiro[imidazolidine-4,3′-indole]-2,2′,5(1′H)-trione | | |
| 110 | 5′-methyl-1′-(4-methyl-3-pentenyl)-2H,5H- | 1.43 | 314.43 |
| | spiro[imidazolidine-4,3′-indole]-2,2′,5(1′H)-trione | | |
| 111 | 1′-{2-[(4-fluorophenyl)oxy]ethyl}-5′-methyl-2H,5H- | 1.47 | 369.26 |
| | spiro[imidazolidine-4,3′-indole]-2,2′,5(1′H)-trione | | |
| 112 | 1′-{[2-fluoro-4-(trifluoromethyl)phenyl]methyl}-5′-methyl- | 1.6 | 407.16 |
| | 2H,5H-spiro[imidazolidine-4,3′-indole]-2,2′,5(1′H)-trione | | |
| 113 | 5′-methyl-1′-({4-[(trifluoromethyl)oxy]phenyl}methyl)- | 1.6 | 405.16 |
| | 2H,5H-spiro[imidazolidine-4,3′-indole]-2,2′,5(1′H)-trione | | |
| 114 | 1′-(1,1′-biphenyl-2-ylmethyl)-5′-methyl-2H,5H- | 1.68 | 398.24 |
| | spiro[imidazolidine-4,3′-indole]-2,2′,5(1′H)-trione | | |
| 115 | 1′-(2,1,3-benzoxadiazol-5-ylmethyl)-5′-methyl-2H,5H- | 1.41 | 363.27 |
| | spiro[imidazolidine-4,3′-indole]-2,2′,5(1′H)-trione | | |
| 116 | 1′-{[2-chloro-5-(trifluoromethyl)phenyl]methyl}-5′-methyl- | 1.63 | 423.09 |
| | 2H,5H-spiro[imidazolidine-4,3′-indole]-2,2′,5(1′H)-trione | | |
| 117 | 5′-methyl-1′-{[6-(trifluoromethyl)-3-pyridinyl]methyl}- | 1.38 | 391.21 |
| | 2H,5H-spiro[imidazolidine-4,3′-indole]-2,2′,5(1′H)-trione | | |
| 118 | 5′-methyl-1′-[3-(methyloxy)propyl]-2H,5H- | 1.08 | 303.43 |
| | spiro[imidazolidine-4,3′-indole]-2,2′,5(1′H)-trione | | |
| 119 | 1′-[(2-chloro-6-fluorophenyl)methyl]-5′-methyl-2H,5H- | 1.47 | 373.21 |
| | spiro[imidazolidine-4,3′-indole]-2,2′,5(1′H)-trione | | |
| 120 | 1′-[(4-chlorophenyl)methyl]-5′-methyl-2H,5H- | 1.52 | 355.27 |
| | spiro[imidazolidine-4,3′-indole]-2,2′,5(1′H)-trione | | |
| 121 | 1′-{[4-(1,1-dimethylethyl)phenyl]methyl}-5′-methyl-2H,5H- | 1.68 | 377.31 |
| | spiro[imidazolidine-4,3′-indole]-2,2′,5(1′H)-trione | | |
| 122 | 5′-methyl-1′-{[4-(trifluoromethyl)phenyl]methyl}-2H,5H- | 1.58 | 389.21 |
| | spiro[imidazolidine-4,3′-indole]-2,2′,5(1′H)-trione | | |
| 123 | 1′-[(2,4-dichlorophenyl)methyl]-5′-methyl-2H,5H- | 1.62 | 389.15 |
| | spiro[imidazolidine-4,3′-indole]-2,2′,5(1′H)-trione | | |
| 124 | 5′-methyl-1′-[2-(1H-pyrrol-1-yl)ethyl]-2H,5H- | 1.3 | 324.38 |
| | spiro[imidazolidine-4,3′-indole]-2,2′,5(1′H)-trione | | |
| 125 | 1′-[(2,3-dichlorophenyl)methyl]-5′-methyl-2H,5H- | 1.6 | 389.16 |
| | spiro[imidazolidine-4,3′-indole]-2,2′,5(1′H)-trione | | |
| 126 | 5′-methyl-1′-(3-methylbutyl)-2H,5H-spiro[imidazolidine-4,3′- | 1.41 | 301.45 |
| | indole]-2,2′,5(1′H)-trione | | |
| 127 | 1′-[(6-chloro-3-pyridinyl)methyl]-5′-methyl-2H,5H- | 1.28 | 356.24 |
| | spiro[imidazolidine-4,3′-indole]-2,2′,5(1′H)-trione | | |
| 128 | 5′-methyl-1′-pentyl-2H,5H-spiro[imidazolidine-4,3′-indole]- | 1.43 | 301.44 |
| | 2,2′,5(1′H)-trione | | |
| 129 | 1′-[(2-iodophenyl)methyl]-5′-methyl-2H,5H- | 1.57 | 446.98 |
| | spiro[imidazolidine-4,3′-indole]-2,2′,5(1′H)-trione | | |
| 130 | 1′-[(4-ethenylphenyl)methyl]-5′-methyl-2H,5H- | 1.53 | 347.34 |
| | spiro[imidazolidine-4,3′-indole]-2,2′,5(1′H)-trione | | |
| 131 | 5′-methyl-1′-(phenylmethyl)-2H,5H-spiro[imidazolidine-4,3′- | 1.4 | 321.37 |
| | indole]-2,2′,5(1′H)-trione | | |
| 132 | 1′-[(2E)-2-butenyl]-5′-methyl-2H,5H-spiro[imidazolidine- | 1.27 | 285.46 |
| | 4,3′-indole]-2,2′,5(1′H)-trione | | |
| 133 | 5′-methyl-1′-[2-(methyloxy)ethyl]-2H,5H- | 1.02 | 289.44 |
| | spiro[imidazolidine-4,3′-indole]-2,2′,5(1′H)-trione | | |
| 134 | 5′-methyl-1′-(2-propenyl)-2H,5H-spiro[imidazolidine-4,3′- | 1.16 | 271.47 |
| | indole]-2,2′,5(1′H)-trione | | |
| 135 | 5′-methyl-1′-[(2E)-3-phenyl-2-propenyl]-2H,5H- | 1.53 | 347.34 |
| | spiro[imidazolidine-4,3′-indole]-2,2′,5(1′H)-trione | | |
| 136 | 1′-[(3,5-dimethyl-4-isoxazolyl)methyl]-5′-methyl-2H,5H- | 1.2 | 340.33 |
| | spiro[imidazolidine-4,3′-indole]-2,2′,5(1′H)-trione | | |
| 137 | 5′-methyl-1′-(1-phenylethyl)-2H,5H-spiro[imidazolidine-4,3′- | 1.47 | 335.36 |
| | indole]-2,2′,5(1′H)-trione | | |
| 138 | 5′-methyl-1′-(2-phenylethyl)-2H,5H-spiro[imidazolidine-4,3′- | 1.45 | 335.34 |
| | indole]-2,2′,5(1′H)-trione | | |
| 139 | 5′-methyl-1′-{[2-(trifluoromethyl)phenyl]methyl}-2H,5H- | 1.58 | 389.2 |
| | spiro[imidazolidine-4,3′-indole]-2,2′,5(1′H)-trione | | |
| 140 | 1′-(2-ethylbutyl)-5′-methyl-2H,5H-spiro[imidazolidine-4,3′- | 1.48 | 315.42 |
| | indole]-2,2′,5(1′H)-trione | | |
| 141 | 1′-{[4-fluoro-3-(trifluoromethyl)phenyl]methyl}-5′-methyl- | 1.57 | 407.16 |
| | 2H,5H-spiro[imidazolidine-4,3′-indole]-2,2′,5(1′H)-trione | | |
| 142 | 5′-methyl-1′-{[4-(methylsulfonyl)phenyl]methyl}-2H,5H- | 1.25 | 399.18 |
| | spiro[imidazolidine-4,3′-indole]-2,2′,5(1′H)-trione | | |
| 143 | 1′-[2-(1H-indol-3-yl)ethyl]-5′-methyl-2H,5H- | 1.48 | 374.29 |
| | spiro[imidazolidine-4,3′-indole]-2,2′,5(1′H)-trione | | |
| 144 | 5′-methyl-1′-{[5-(trifluoromethyl)-2-furanyl]methyl}-2H,5H- | 1.5 | 379.22 |
| | spiro[imidazolidine-4,3′-indole]-2,2′,5(1′H)-trione | | |
| 145 | 5′-methyl-1′-[(2,4,6-trimethylphenyl)methyl]-2H,5H- | 1.62 | 363.34 |
| | spiro[imidazolidine-4,3′-indole]-2,2′,5(1′H)-trione | | |
| 146 | 1′-(cyclopropylmethyl)-5′-methyl-2H,5H- | 1.23 | 285.46 |
| | spiro[imidazolidine-4,3′-indole]-2,2′,5(1′H)-trione | | |
| 147 | 5′-methyl-1′-[2-(phenyloxy)ethyl]-2H,5H- | 1.45 | 351.31 |
| | spiro[imidazolidine-4,3′-indole]-2,2′,5(1′H)-trione | | |
| 148 | 5′-methyl-1′-{[3-(trifluoromethyl)phenyl]methyl}-2H,5H- | 1.55 | 389.22 |
| | spiro[imidazolidine-4,3′-indole]-2,2′,5(1′H)-trione | | |
| 149 | 5′-methyl-1′-({3-[(trifluoromethyl)oxy]phenyl}methyl)- | 1.58 | 405.18 |
| | 2H,5H-spiro[imidazolidine-4,3′-indole]-2,2′,5(1′H)-trione | | |
| 150 | 5′-methyl-1′-[(5-methyl-3-isoxazolyl)methyl]-2H,5H- | 1.22 | 326.35 |
| | spiro[imidazolidine-4,3′-indole]-2,2′,5(1′H)-trione | | |
| 151 | 5′-methyl-1′-(2-{[2-(methyloxy)phenyl]oxy}ethyl)-2H,5H- | 1.43 | 382.27 |
| | spiro[imidazolidine-4,3′-indole]-2,2′,5(1′H)-trione | | |
| 152 | 1′-[(2-chloro-1,3-thiazol-5-yl)methyl]-5′-methyl-2H,5H- | 1.28 | 362.2 |
| | spiro[imidazolidine-4,3′-indole]-2,2′,5(1′H)-trione | | |
| 153 | 1′-{[5-(4-chlorophenyl)-1,3,4-oxadiazol-2-yl]methyl}-5′- | 1.48 | 423.11 |
| | methyl-2H,5H-spiro[imidazolidine-4,3′-indole]-2,2′,5(1′H)- | | |
| | trione | | |
| 154 | 5′-methyl-1′-({5-[4-(methyloxy)phenyl]-1,3,4-oxadiazol-2- | 1.38 | 420.17 |
| | yl}methyl)-2H,5H-spiro[imidazolidine-4,3′-indole]- | | |
| | 2,2′,5(1′H)-trione | | |
| 155 | 1′-(cyclohexylmethyl)-5′-methyl-2H,5H-spiro[imidazolidine- | 1.52 | 327.41 |
| | 4,3′-indole]-2,2′,5(1′H)-trione | | |
| 156 | 1′-(cyclobutylmethyl)-5′-methyl-2H,5H-spiro[imidazolidine- | 1.33 | 299.44 |
| | 4,3′-indole]-2,2′,5(1′H)-trione | | |
| 157 | 1′-[2-(ethyloxy)ethyl]-5′-methyl-2H,5H-spiro[imidazolidine- | 1.12 | 303.42 |
| | 4,3′-indole]-2,2′,5(1′H)-trione | | |
| 158 | 1′-[(4-bromo-2-fluorophenyl)methyl]-5′-methyl-2H,5H- | 1.57 | 417.06 |
| | spiro[imidazolidine-4,3′-indole]-2,2′,5(1′H)-trione | | |
| 159 | 5′-methyl-1′-{[4-(1,2,3-thiadiazol-4-yl)phenyl]methyl}- | 1.45 | 405.17 |
| | 2H,5H-spiro[imidazolidine-4,3′-indole]-2,2′,5(1′H)-trione | | |
| 160 | 1′-(tetrahydro-2H-pyran-2-ylmethyl)-2H,5H- | 1.13 | 315.4 |
| | spiro[imidazolidine-4,3′-indole]-2,2′,5(1′H)-trione | | |
| 161 | 1′-(2-propynyl)-2H,5H-spiro[imidazolidine-4,3′-indole]- | 0.88 | 255.49 |
| | 2,2′,5(1′H)-trione | | |
| 162 | 5′-chloro-1′-(4-methyl-3-pentenyl)-2H,5H- | 1.48 | 333.32 |
| | spiro[imidazolidine-4,3′-indole]-2,2′,5(1′H)-trione | | |
| 163 | 5′-chloro-1′-[2-(phenyloxy)ethyl]-2H,5H- | 1.5 | 371.21 |
| | spiro[imidazolidine-4,3′-indole]-2,2′,5(1′H)-trione | | |
| 164 | 5′-chloro-1′-[2-(1H-pyrrol-1-yl)ethyl]-2H,5H- | 1.35 | 344.26 |
| | spiro[imidazolidine-4,3′-indole]-2,2′,5(1′H)-trione | | |
| 165 | 5′-chloro-1′-[(2-chloro-1,3-thiazol-5-yl)methyl]-2H,5H- | 1.33 | 382.08 |
| | spiro[imidazolidine-4,3′-indole]-2,2′,5(1′H)-trione | | |
| 166 | 5′-chloro-1′-(cyclohexylmethyl)-2H,5H-spiro[imidazolidine- | 1.55 | 347.32 |
| | 4,3′-indole]-2,2′,5(1′H)-trione | | |
| 167 | 5′-chloro-1′-(cyclobutylmethyl)-2H,5H-spiro[imidazolidine- | 1.4 | 319.35 |
| | 4,3′-indole]-2,2′,5(1′H)-trione | | |
| 168 | 5′-chloro-1′-[2-(ethyloxy)ethyl]-2H,5H-spiro[imidazolidine- | 1.16 | 323.34 |
| | 4,3′-indole]-2,2′,5(1′H)-trione | | |
| 169 | 5′-chloro-1′-(phenylmethyl)-2H,5H-spiro[imidazolidine-4,3′- | 1.45 | 341.29 |
| | indole]-2,2′,5(1′H)-trione | | |
| 170 | 5′-chloro-1′-[2-(methyloxy)ethyl]-2H,5H- | 1.05 | 309.35 |
| | spiro[imidazolidine-4,3′-indole]-2,2′,5(1′H)-trione | | |
| 171 | 5′-chloro-1′-[(3,5-dimethyl-4-isoxazolyl)methyl]-2H,5H- | 1.27 | 360.24 |
| | spiro[imidazolidine-4,3′-indole]-2,2′,5(1′H)-trione | | |
| 172 | 5′-chloro-1′-pentyl-2H,5H-spiro[imidazolidine-4,3′-indole]- | 1.47 | 321.36 |
| | 2,2′,5(1′H)-trione | | |
| 173 | 5′-fluoro-1′-(imidazo[1,2-a]pyridin-2-ylmethyl)-2H,5H- | 0.92 | 365.25 |
| | spiro[imidazolidine-4,3′-indole]-2,2′,5(1′H)-trione | | |
| 174 | 1′-{[5-(4-chlorophenyl)-1,3,4-oxadiazol-2-yl]methyl}-5′- | 1.47 | 427.06 |
| | fluoro-2H,5H-spiro[imidazolidine-4,3′-indole]-2,2′,5(1′H)- | | |
| | trione | | |
| 175 | 5′-fluoro-1′-(3-pyridinylmethyl)-2H,5H-spiro[imidazolidine- | 0.58 | 326.33 |
| | 4,3′-indole]-2,2′,5(1′H)-trione | | |
| 176 | 5′-fluoro-1′-(tetrahydro-2H-pyran-2-ylmethyl)-2H,5H- | 1.22 | 333.35 |
| | spiro[imidazolidine-4,3′-indole]-2,2′,5(1′H)-trione | | |
| 177 | 1′-(2,1,3-benzoxadiazol-5-ylmethyl)-5′-fluoro-2H,5H- | 1.37 | 367.22 |
| | spiro[imidazolidine-4,3′-indole]-2,2′,5(1′H)-trione | | |
| 178 | 5′-fluoro-1′-{[2-fluoro-4-(trifluoromethyl)phenyl]methyl}- | 1.57 | 411.12 |
| | 2H,5H-spiro[imidazolidine-4,3′-indole]-2,2′,5(1′H)-trione | | |
| 179 | 5′-fluoro-1′-{[4-fluoro-3-(trifluoromethyl)phenyl]methyl}- | 1.55 | 411.12 |
| | 2H,5H-spiro[imidazolidine-4,3′-indole]-2,2′,5(1′H)-trione | | |
| 180 | 5′-chloro-7′-methyl-1′-(2-methylpropyl)-2H,5H- | 1.43 | 321.34 |
| | spiro[imidazolidine-4,3′-indole]-2,2′,5(1′H)-trione | | |
| 181 | 5′-chloro-7′-methyl-1′-(2-phenylethyl)-2H,5H- | 1.57 | 369.23 |
| | spiro[imidazolidine-4,3′-indole]-2,2′,5(1′H)-trione | | |
| 182 | 5′-chloro-7′-methyl-1′-(4-methyl-3-pentenyl)-2H,5H- | 1.57 | 347.3 |
| | spiro[imidazolidine-4,3′-indole]-2,2′,5(1′H)-trione | | |
| 183 | 5′-chloro-1′-(2-ethylbutyl)-7′-methyl-2H,5H- | 1.6 | 349.31 |
| | spiro[imidazolidine-4,3′-indole]-2,2′,5(1′H)-trione | | |
| 184 | 1′-(2,1,3-benzoxadiazol-5-ylmethyl)-5′-chloro-7′-methyl- | 1.53 | 397.87 |
| | 2H,5H-spiro[imidazolidine-4,3′-indole]-2,2′,5(1′H)-trione | | |
| 185 | 5′-chloro-7′-methyl-1′-{[6-(trifluoromethyl)-3- | 1.48 | 424.07 |
| | pyridinyl]methyl}-2H,5H-spiro[imidazolidine-4,3′-indole]- | | |
| | 2,2′,5(1′H)-trione | | |
| 186 | 5′-chloro-7′-methyl-1′-[(5-methyl-3-isoxazolyl)methyl]- | 1.4 | 360.24 |
| | 2H,5H-spiro[imidazolidine-4,3′-indole]-2,2′,5(1′H)-trione | | |
| 187 | 5′-chloro-7′-methyl-1′-(2-{[2-(methyloxy)phenyl]oxy}ethyl)- | 1.57 | 415.13 |
| | 2H,5H-spiro[imidazolidine-4,3′-indole]-2,2′,5(1′H)-trione | | |
| 188 | 5′-chloro-1′-[(2-chloro-l,3-thiazol-5-yl)methyl]-7′-methyl- | 1.41 | 396.07 |
| | 2H,5H-spiro[imidazolidine-4,3′-indole]-2,2′,5(1′H)-trione | | |
| 189 | 5′-chloro-7′-methyl-1′-[(2E)-3-phenyl-2-propenyl]-2H,5H- | 1.62 | 381.2 |
| | spiro[imidazolidine-4,3′-indole]-2,2′,5(1′H)-trione | | |
| 190 | 1′-[(2,6-dichloro-4-pyridinyl)methyl]-5′-methyl-2H,5H- | 1.45 | 390.14 |
| | spiro[imidazolidine-4,3′-indole]-2,2′,5(1′H)-trione | | |
| 191 | 5′-methyl-1′-(tetrahydro-2H-pyran-2-ylmethyl)-2H,5H- | 1.26 | 329.39 |
| | spiro[imidazolidine-4,3′-indole]-2,2′,5(1′H)-trione | | |
| 192 | 5′-methyl-1′-(2-propynyl)-2H,5H-spiro[imidazolidine-4,3′- | 1.12 | 269.47 |
| | indole]-2,2′,5(1′H)-trione | | |
| 193 | 1′-(imidazo[1,2-a]pyridin-2-ylmethyl)-5′-methyl-2H,5H- | 0.98 | 361.3 |
| | spiro[imidazolidine-4,3′-indole]-2,2′,5(1′H)-trione | | |

### Pharmacology

1. hVR1 FLIPR (Fluorometric Image Plate Reader) Screening Assay

Transfected CHO cells, stably expressing hVR1 (15,000 cells/well) are seeded in 50 μL media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 min dye incubation in the dark at 37° C. and 2% CO₂, the extracellular dye present is washed away using an EMBLA (Scatron), leaving the cells in 40 μL of assay buffer (1×HBSS, 10 mM D-Glucose, 1 mM CaCl₂, 10 mM HEPES, 10×7.5% NaHCO₃ and 2.5 mM Probenecid).

### FLIPR Assay—IC₅₀ Determination Protocol

For IC₅₀ determinations the fluorescence is read using FLIPR filter 1 (em 520-545 nM). A cellular baseline recording is taken for 30 seconds, followed by a 20 μL addition of 10, titrated half-log concentrations of the test compound, yielding cellular concentration ranging from 3 μM to 0.1 nM. Data is collected every 2 seconds for a further 5 min prior to the addition of a VR1 agonist solution: either 50 nM solution of capsaicin or MES (2-[N-morpholino]ethanesulfonic acid) buffer (pH 5.2), by the FLIPR pipettor. The FLIPR continues to collect data for a further 4 min. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated.

### LIST OF ABBREVIATIONS

VR1 vanilloid receptor 1
IBS irritable bowel syndrome
IBD inflammatory bowel disease
GERD gastro-esophageal reflux disease
HEPES 4-(2-Hydroxyethyl)piperazine-1-ethanesulfonic acid

### Results

Typical IC₅₀ values as measured in the assays described above are 10 μM or less. In one aspect of the invention the IC₅₀ is below 5000 nM. In another aspect of the invention the IC₅₀ is below 3000 nM

TABLE 1
Specimen results from the hVR1 FLIPR.

| Example No. | IC₅₀ nM |
|---|---|
| Compound 3 | 3000 |
| Compound 7 | 3010 |
| Compound 5 | 2110 |

1-26. (canceled)

27. A compound having the formula I 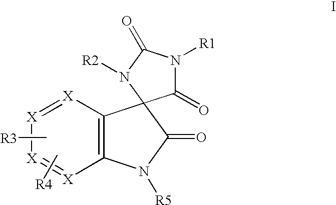
wherein:
R¹ is selected from H, C₁₋₁₀alkyl, C₂₋₁₀alkenyl, C₂₋₁₀alkynyl, C₃₋₁₀cycloalkyl, C₃₋₁₀)cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, C₃₋₅heteroaryl, C₆₋₁₀aryl and C₃₋₆heterocycloalkyl, C₃₋₆heteroaryl-C₁₋₆alkyl, C₆₋₁₀aryl-C₁₋₆alkyl and C₁₋₆ alkyl-oxy-C₁₋₅alkyl, whereby R¹ may optionally be substituted by one or more groups selected from halogen, cyano, nitro, methoxy, ethoxy, methyl, ethyl, hydroxy and —NR⁶R⁷;
R² is selected from H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, C₃₋₅heteroaryl, C₆₋₁₀aryl, C₃₋₆heterocycloalkyl, C₃₋₆heteroaryl-C₁₋₆alkyl, C₆₋₁₀aryl-C₁₋₆alkyl and C₁₋₆ alkyl-oxy-C₁₋₅alkyl, whereby R² may optionally be substituted by one or more groups selected from halogen, cyano, nitro, methoxy, ethoxy, methyl, ethyl, hydroxy and —NR⁶R⁷;
R³ is selected from H, halogen, C₁₋₁₀alkyl, haloalkyl, haloalkylO, C₂₋₁₀alkenyl, C₂₋₁₀alkynyl, C₃₋₁₀cycloalkyl, C₃₋₁₀cycloalkyl-C₁₋₆alkyl and C₄₋₈cycloalkenyl-C₁₋₆alkyl;
R⁴ is selected from H, halogen, haloalkyl, haloalkylO, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl and C₄₋₈cycloalkenyl-C₁₋₆alkyl;
R⁵ is selected from C₂₋₁₀alkenyl, C₂₋₁₀alkynyl, C₃₋₁₀cycloalkyl, C₃₋₁₀cycloalkylC₁₋₆alkyl, C₄₋₈cycloalkenyl, C₄₋₈cycloalkenyl-C₁₋₆alkyl, C₃₋₆heterocycloalkyl-C₁₋₆alkyl, C₃₋₅heteroaryl, C₃₋₆heterocycloalkyl, C₁₋₆ alkyl-oxy-C₁₋₅alkyl, C₂₋₆ alkenyl-oxy-C₁₋₆alkyl, C₂₋₆ alkynyl-oxy-C₁₋₆alkyl, C₆₋₁₀aryl-oxy-C₁₋₆ alkyl, C₁₋₆ alkyl-O—C₅₋₁₀heteroaryl, C₆₋₁₀aryl-C₁₋₆alkyl, C₃₋₆heteroaryl-C₁₋₆alkyl, C₆₋₁₀aryl-C₂₋₆alkenyl, C₆₋₁₀aryl-C₂₋₆alkynyl, C₃₋₆heteroaryl-C₂₋₆alkenyl, C₃₋₆heteroaryl-C₂₋₆alkynyl, R⁶C(═O)N(—R⁷)—C₁₋₆alkyl, R⁶R⁷N—, R⁶R⁷N—C(═O)—C₁₋₆alkyl, R⁶R⁷NS(═O)₂—C₁₋₆alkyl, R⁶CS(═O)₂N(—R⁶)—C₁₋₆alkyl, R⁶R⁷NC(═O)N(—R⁸)—C₁₋₆alkyl, R⁶R⁷NC(═O)N(—R⁸)—C₁₋₆alkyl and R⁶R⁷NS(═O)₂N(R⁸)—C₁₋₆alkyl,
whereby any C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocycloalkyl-C₁₋₆alkyl, R⁶R⁷N—, C₃₋₅heteroaryl, C₆₋₁₀aryl, C₃₋₆heterocycloalkyl, C₁₋₆ alkyl-oxy-C₁₋₅alkyl, C₂₋₆ alkenyl-oxy-C₁₋₆alkyl, C₂₋₆ alkynyl-oxy-C₁₋₆alkyl, C₁₋₆ alkyl-oxy-C₆₋₁₀aryl, C₁₋₆ alkyl-oxy-C₅₋₁₀heteroaryl or C₆₋₁₀aryl-C₁₋₆alkyl, C₅₋₁₀heteroaryl-C₁₋₆alkyl, C₆₋₁₀aryl-C₂₋₆alkenyl, C₆₋₁₀aryl-C₂₋₆alkynyl, C₅₋₁₀heteroaryl-C₂₋₆alkenyl, C₆₋₁₀heteroaryl-C₂₋₆alkynyl, R⁶C(═O)N(—R⁷)—C₁₋₆alkyl and R⁶R⁷N—C(═O)—C₁₋₆alkyl, may optionally be substituted by one or more groups selected from halogen, cyano, nitro, CF₃, OCF₃, trimethylsilyl, hydroxy, —NR⁶R⁷, SO₂R⁷, R⁶O—C₁₋₆ alkyl, C₁₋₆alkyl, C₂₋₆alkenyl, C₂₋₆alkynyl, C₆₋₁₀aryl and C₅₋₁₀heteroaryl;
R⁶, R⁷ and R⁸ are independently selected from H, C₁₋₆alkyl, C₂₋₆alkenyl, C₂₋₆alkynyl, substituted or unsubstituted C₆₋₁₀aryl, substituted or unsubstituted C₃₋₆heteroaryl and a divalent C₂₋₆group that together with another divalent R⁵, R⁶ or R⁷ forms a portion of a ring;
X is selected from N, CH and CR⁹,
whereby R⁹ is selected from H, halogen, haloalkyl, haloalkylO, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;
or salts thereof,
with the proviso that R⁵ is not a naphthylmethyl or cinnamyl radical,
and with the proviso that the compound does not have the formula III: 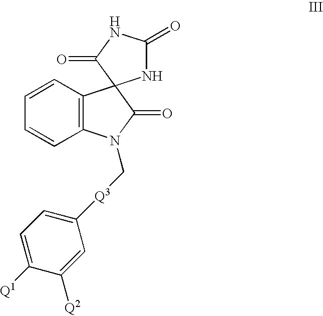
where Q¹ and Q² are independently halo or C₁₋₃haloalkyl and
Q³ is ethenyl or ethynyl.

28. A compound according to claim 27, wherein:
R¹ is H, C₁₋₁₀alkyl or C₁₋₆ alkyl-oxy-C₁₋₅alkyl;
R² is H, C₁₋₁₀alkyl or C₁₋₆ alkyl-oxy-C₁₋₅alkyl;
R³ is H, halogen, C₁₋₁₀alkyl or haloalkylO;
R⁴ is H, halogen, haloalkylO or C₁₋₁₀alkyl;
R⁵ is C₂₋₁₀alkenyl, C₂₋₁₀alkynyl, C₃₋₁₀cycloalkyl, C₃₋₁₀cycloalkyl-C₁₋₆alkyl, C₃₋₆heterocycloalkyl-C₁₋₆alkyl, C₁₋₆ alkyl-oxy-C₁₋₅alkyl, C₆₋₁₀aryl-oxy-C₁₋₆ alkyl, C₆₋₁₀arylC₁₋₆alkyl, C₃₋₆heteroaryl-C₁₋₆alkyl, C₆₋₁₀aryl-C₂₋₆alkenyl, C₆₋₁₀aryl-C₂₋₆alkynyl, C₃₋₆heteroaryl-C₂₋₆alkenyl, C₃₋₆heteroaryl-C₂₋₆alkynyl or R⁶R⁷N—C(═O)—C₁₋₆alkyl,
whereby any C₁₋₁₀alkyl, C₃₋₁₀cycloalkyl, C₃₋₁₀cycloalkyl-C₁₋₆alkyl, C₃₋₆heterocycloalkyl-C₁₋₆alkyl, C₃₋₅heteroaryl, C₆₋₁₀aryl, C₅₋₁₀heteroaryl-C₁₋₆alkyl and R⁶R⁷N—C(═O)—C₁₋₆alkyl, may optionally be substituted by one or more groups selected from CF₃, methoxy, ethoxy, OCF₃, methyl, tert-butyl, SO₂R⁷, R⁶O—C₁₋₆ alkyl, C₁₋₆alkyl, C₂₋₆alkenyl, C₆₋₁₀aryl and C₅₋₁₀heteroaryl;
R⁶, R⁷ and R⁸ are independently selected from H, C₁₋₆alkyl, substituted or unsubstituted C₆₋₁₀aryl and substituted and unsubstituted C₃₋₆heteroaryl;
X is selected from N, CH and CR⁹,
wherein R⁹ is selected from H, halogen, haloalkylO and C₁₋₁₀alkyl.

29. A compound having the formula II, 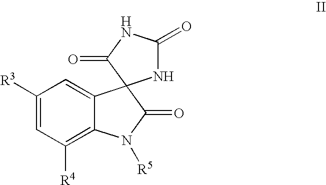
wherein R³ to R⁹ are as defined as in claim 27,
with the proviso that the compound does not have the formula III: 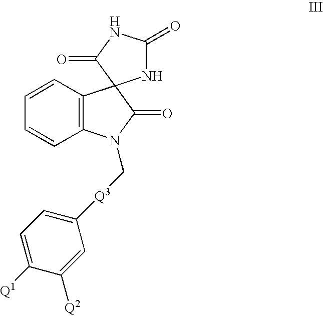
where Q¹ and Q² are independently halo or C₁₋₃haloalkyl and
Q³ is ethenyl or ethynyl.

30. The compound according to claim 27, wherein R³ is hydrogen, bromo, chloro, fluoro, methyl, ethyl, propyl or fluoromethyl, difluoromethyl, trifluoromethyl, fluoromethoxy, difluoromethoxy or trifluoromethoxy.

31. The compound according to claim 27, wherein X is CH.

32. A compound selected from:
1′-[(2,6-dichloro-4-pyridinyl)methyl]-2H,5H-spiro[imidazolidine-4,3′-indole]-2,2′,5 (1′H)-trione,
1′-[2-(H-indol-3-yl)ethyl]-2H,5H-spiro[imidazolidine-4,3′-indole]-2,2′,5 (1′H)-trione,
1′-[3-(methyloxy)propyl]-2H,5H-spiro[imidazolidine-4,3′-indole]-2,2′,5 (1′H)-trione,
1′-(cyclopropylmethyl)-2H,5H-spiro[imidazolidine-4,3′-indole]-2,2′,5(1′H)-trione,
1′-(2-{[2-(methyloxy)phenyl]oxy}ethyl)-2H,5H-spiro[imidazolidine-4,3′-indole]-2,2′,5(1′H)-trione,
1′-[(2-chloro-1,3-thiazol-5-yl)methyl]-2H,5H-spiro[imidazolidine-4,3′-indole]-2,2′,5(1′H)-trione,
1′-{[5-(4-chlorophenyl)-1,3,4-oxadiazol-2-yl]methyl}-2H,5H-spiro[imidazolidine-4,3′-indole]-2,2′,5(1′H)-trione,
1′-(cyclohexylmethyl)-2H,5H-spiro[imidazolidine-4,3′-indole]-2,2′,5(1′H)-trione,
1′-[2-(methyloxy)ethyl]-2H,5H-spiro[imidazolidine-4,3′-indole]-2,2′,5 (1′H)-trione,
5′-chloro-1′-(2-phenylethyl)-2H,5H-spiro[imidazolidine-4,3′-indole]-2,2′,5(1′H)-trione,
5′-chloro-1′-(2-{[2-(methyloxy)phenyl]oxy}ethyl)-2H,5H-spiro[imidazolidine-4,3′-indole]-2,2′,5(1′H)-trione,
5′-chloro-1′-[(6-chloro-3-pyridinyl)methyl]-2H,5H-spiro[imidazolidine-4,3′-indole]-2,2′,5(1′H)-trione,
5′-fluoro-1′-(2-phenylethyl)-2H,5H-spiro[imidazolidine-4,3′-indole]-2,2′,5 (1′H)-trione,
5′-fluoro-1′-{2-[(4-fluorophenyl)oxy]ethyl}-2H,5H-spiro[imidazolidine-4,3′-indole]-2,2′,5(1′H)-trione,
1′-[(2,6-dichloro-4-pyridinyl)methyl]-5′-fluoro-2H,5H-spiro[imidazolidine-4,3′-indole]-2,2′,5(1′H)-trione,
5′-fluoro-1′-[2-(1H-indol-3-yl)ethyl]-2H,5H-spiro[imidazolidine-4,3′-indole]-2,2′,5(1′H)-trione,
5′-fluoro-1′-{[6-(trifluoromethyl)-3-pyridinyl]methyl}-2H,5H-spiro[imidazolidine-4,3′-indole]-2,2′,5(1′H)-trione,
5′-fluoro-1′-[3-(methyloxy)propyl]-2H,5H-spiro[imidazolidine-4,3′-indole]-2,2′,5(1′H)-trione,
5′-fluoro-1′-{[5-(trifluoromethyl)-2-furanyl]methyl}-2H,5H-spiro[imidazolidine-4,3′-indole]-2,2′,5(1′H)-trione,
1′-(cyclopropylmethyl)-5′-fluoro-2H,5H-spiro[imidazolidine-4,3′-indole]-2,2′,5(1′H)-trione,
5′-fluoro-1′-[2-(phenyloxy)ethyl]-2H,5H-spiro[imidazolidine-4,3′-indole]-2,2′,5(1′H)-trione,
5′-fluoro-1′-[2-(1H-pyrrol-1-yl)ethyl]-2H,5H-spiro[imidazolidine-4,3′-indole]-2,2′,5(1′H)-trione,
5′-fluoro-1′-[(5-methyl-3-isoxazolyl)methyl]-2H,5H-spiro[imidazolidine-4,3′-indole]-2,2′,5(1′H)-trione,
5′-fluoro-1′-(2-{[2-(methyloxy)phenyl]oxy}ethyl)-2H,5H-spiro[imidazolidine-4,3′-indole]-2,2′,5(1′H)-trione,
1′-[(2-chloro-1,3-thiazol-5-yl)methyl]-5′-fluoro-2H,5H-spiro[imidazolidine-4,3′-indole]-2,2′,5(1′ H)-trione,
5′-fluoro-1′-({5-[4-(methyloxy)phenyl]-1,3,4-oxadiazol-2-yl}methyl)-2H,5H-spiro[imidazolidine-4,3′-indole]-2,2′,5(1′H)-trione,
1′-(cyclohexylmethyl)-5′-fluoro-2H,5H-spiro[imidazolidine-4,3′-indole]-2,2′,5(1′H)-trione,
1′-[(6-chloro-3-pyridinyl)methyl]-5′-fluoro-2H,5H-spiro[imidazolidine-4,3′-indole]-2,2′,5(1′H)-tri one,
5′-fluoro-1′-(2-propynyl)-2H,5H-spiro[imidazolidine-4,3′-indole]-2,2′,5 (1′H)-trione,
1′-(cyclobutylmethyl)-5′-fluoro-2H,5H-spiro[imidazolidine-4,3′-indole]-2,2′,5(1′H)-trione,
1′-[2-(ethyloxy)ethyl]-5′-fluoro-2H,5H-spiro[imidazolidine-4,3′-indole]-2,2′,5(1′H)-trione,
5′-fluoro-1′-[2-(methyloxy)ethyl]-2H,5H-spiro[imidazolidine-4,3′-indole]-2,2′,5(1′H)-trione,
1′-[(3,5-dimethyl-4-isoxazolyl)methyl]-5′-fluoro-2H,5H-spiro[imidazolidine-4,3′-indole]-2,2′,5(1′H)-trione,
5′-chloro-1′-{2-[(4-fluorophenyl)oxy]ethyl}-7′-methyl-2H,5H-spiro[imidazolidine-4,3′-indole]-2,2′,5(1′H)-trione,
5′-chloro-7′-methyl-1′-[2-(1H-pyrrol-1-yl)ethyl]-2H,5H-spiro[imidazolidine-4,3′-indole]-2,2′,5(1′H)-trione,
5′-chloro-1′-(imidazo[1,2-a]pyridin-2-ylmethyl)-7′-methyl-2H,5H-spiro[imidazolidine-4,3′-indole]-2,2′,5(1′H)-trione,
5′-chloro-1′-(cyclohexylmethyl)-7′-methyl-2H,5H-spiro[imidazolidine-4,3′-indole]-2,2′,5(1′H)-trione,
5′-chloro-1′-[(6-chloro-3-pyridinyl)methyl]-7′-methyl-2H,5H-spiro[imidazolidine-4,3′-indole]-2,2′,5(1′H)-trione,
5′-chloro-1′-(cyclobutylmethyl)-7′-methyl-2H,5H-spiro[imidazolidine-4,3′-indole]-2,2′,5(1′H)-trione,
5′-chloro-1′-[2-(ethyloxy)ethyl]-7′-methyl-2H,5H-spiro[imidazolidine-4,3′-indole]-2,2′,5(1′H)-trione,
5′-chloro-7′-methyl-1′-[2-(methyloxy)ethyl]-2H,5H-spiro[imidazolidine-4,3′-indole]-2,2′,5(1′H)-trione,
5′-chloro-1′-[(3,5-dimethyl-4-isoxazolyl)methyl]-7′-methyl-2H,5H-spiro[imidazolidine-4,3′-indole]-2,2′,5(1′H)-trione,
1′-(2,1,3-benzoxadiazol-5-ylmethyl)-2H,5H-spiro[imidazolidine-4,3′-indole]-2,2′,5 (1′H)-trione,
1′-[2-(1H-pyrrol-1-yl)ethyl]-2H,5H-spiro[imidazolidine-4,3′-indole]-2,2′,5(1′H)-trione,
1′-[(6-chloro-3-pyridinyl)methyl]-2H,5H-spiro[imidazolidine-4,3′-indole]-2,2′,5(1′H)-trione,
1′-pentyl-2H,5H-spiro[imidazolidine-4,3′-indole]-2,2′,5(1′H)-trione,
1′-(cyclobutylmethyl)-2H,5H-spiro[imidazolidine-4,3′-indole]-2,2′,5(1′H)-trione,
1′-[2-(ethyloxy)ethyl]-2H,5H-spiro[imidazolidine-4,3′-indole]-2,2′,5(1′H)-trione,
1′-[(3,5-dimethyl-4-isoxazolyl)methyl]-2H,5H-spiro[imidazolidine-4,3′-indole]-2,2′,5(1′H)-trione,
1′-{2-[(4-fluorophenyl)oxy]ethyl}-5′-methyl-2H,5H-spiro[imidazolidine-4,3′-indole]-2,2′,5(1′H)-trione,
1′-(2,1,3-benzoxadiazol-5-ylmethyl)-5′-methyl-2H,5H-spiro[imidazolidine-4,3′-indole]-2,2′,5(1′H)-trione,
5′-methyl-1′-{[6-(trifluoromethyl)-3-pyridinyl]methyl}-2H,5H-spiro[imidazolidine-4,3′-indole]-2,2′,5(1′H)-trione,
5′-methyl-1′-[3-(methyloxy)propyl]-2H,5H-spiro[imidazolidine-4,3′-indole]-2,2′,5 (1′H)-trione,
5′-methyl-1′-[2-(1H-pyrrol-1-yl)ethyl]-2H,5H-spiro[imidazolidine-4,3′-indole]-2,2′,5(1′H)-trione,
1′-[(6-chloro-3-pyridinyl)methyl]-5′-methyl-2H,5H-spiro[imidazolidine-4,3′-indole]-2,2′,5(1′H)-trione,
5′-methyl-1′-[2-(methyloxy)ethyl]-2H,5H-spiro[imidazolidine-4,3′-indole]-2,2′,5(1′H)-trione,
1′-[(3,5-dimethyl-4-isoxazolyl)methyl]-5′-methyl-2H,5H-spiro[imidazolidine-4,3′-indole]-2,2′,5(1′H)-trione,
5′-methyl-1′-(1-phenylethyl)-2H,5H-spiro[imidazolidine-4,3′-indole]-2,2′,5 (1′H)-trione,
5′-methyl-1′-(2-phenylethyl)-2H,5H-spiro[imidazolidine-4,3′-indole]-2,2′,5 (1′H)-trione,
1′-[2-(1H-indol-3-yl)ethyl]-5′-methyl-2H,5H-spiro[imidazolidine-4,3′-indole]-2,2′,5(1′H)-trione,
5′-methyl-1′-{[5-(trifluoromethyl)-2-furanyl]methyl}-2H,5H-spiro[imidazolidine-4,3′-indole]-2,2′,5(1′H)-trione,
1′-(cyclopropylmethyl)-5′-methyl-2H,5H-spiro[imidazolidine-4,3′-indole]-2,2′,5 (1′H)-trione,
5′-methyl-1′-[2-(phenyloxy)ethyl]-2H,5H-spiro[imidazolidine-4,3′-indole]-2,2′,5 (1′H)-trione,
5′-methyl-1′-[(5-methyl-3-isoxazolyl)methyl]-2H,5H-spiro[imidazolidine-4,3′-indole]-2,2′,5(1′H)-trione,
5′-methyl-1′-(2-{[2-(methyloxy)phenyl]oxy}ethyl)-2H,5H-spiro[imidazolidine-4,3′-indole]-2,2′,5(1′H)-trione,
1′-[(2-chloro-1,3-thiazol-5-yl)methyl]-5′-methyl-2H,5H-spiro[imidazolidine-4,3′-indole]-2,2′,5(1′H)-trione,
1′-{[5-(4-chlorophenyl)-1,3,4-oxadiazol-2-yl]methyl}-5′-methyl-2H,5H-spiro[imidazolidine-4,3′-indole]-2,2′,5(1′H)-trione,
5′-methyl-1′-({5-[4-(methyloxy)phenyl]-1,3,4-oxadiazol-2-yl}methyl)-2H,5H-spiro[imidazolidine-4,3′-indole]-2,2′,5(1′H)-trione,
1′-(cyclohexylmethyl)-5′-methyl-2H,5H-spiro[imidazolidine-4,3′-indole]-2,2′,5(1′H)-trione,
1′-(cyclobutylmethyl)-5′-methyl-2H,5H-spiro[imidazolidine-4,3′-indole]-2,2′,5(1′H)-trione,
1′-[2-(ethyloxy)ethyl]-5′-methyl-2H,5H-spiro[imidazolidine-4,3′-indole]-2,2′,5 (1′H)-trione,
1′-(tetrahydro-2H-pyran-2-ylmethyl)-2H,5H-spiro[imidazolidine-4,3′-indole]-2,2′,5(1′H)-trione,
1′-(2-propynyl)-2H,5H-spiro[imidazolidine-4,3′-indole]-2,2′,5 (1′H)-trione,
5′-chloro-1′-[2-(phenyloxy)ethyl]-2H,5H-spiro[imidazolidine-4,3′-indole]-2,2′,5(1′H)-trione,
5′-chloro-1′-[2-(1H-pyrrol-1-yl)ethyl]-2H,5H-spiro[imidazolidine-4,3′-indole]-2,2′,5(1′H)-trione,
5′-chloro-1′-[(2-chloro-1,3-thiazol-5-yl)methyl]-2H,5H-spiro[imidazolidine-4,3′-indole]-2,2′,5(1′H)-trione,
5′-chloro-1′-(cyclohexylmethyl)-2H,5H-spiro[imidazolidine-4,3′-indole]-2,2′,5 (1′H)-trione,
5′-chloro-1′-(cyclobutylmethyl)-2H,5H-spiro[imidazolidine-4,3′-indole]-2,2′,5(1′H)-trione,
5′-chloro-1′-[2-(ethyloxy)ethyl]-2H,5H-spiro[imidazolidine-4,3′-indole]-2,2′,5(1′H)-trione,
5′-chloro-1′-[2-(methyloxy)ethyl]-2H,5H-spiro[imidazolidine-4,3′-indole]-2,2′,5(1′H)-trione,
5′-chloro-1′-[(3,5-dimethyl-4-isoxazolyl)methyl]-2H,5H-spiro[imidazolidine-4,3′-indole]-2,2′,5(1′H)-trione,
5′-fluoro-1′-(imidazo[1,2-a]pyridin-2-ylmethyl)-2H,5H-spiro[imidazolidine-4,3′-indole]-2,2′,5(1′H)-trione,
1′-{[5-(4-chlorophenyl)-1,3,4-oxadiazol-2-yl]methyl}-5′-fluoro-2H,5H-spiro[imidazolidine-4,3′-indole]-2,2′,5(1′H)-trione,
5′-fluoro-1′-(3-pyridinylmethyl)-2H,5H-spiro[imidazolidine-4,3′-indole]-2,2′,5 (1′H)-trione,
5′-fluoro-1′-(tetrahydro-2H-pyran-2-ylmethyl)-2H,5H-spiro[imidazolidine-4,3′-indole]-2,2′,5(1′H)-trione,
1′-(2,1,3-benzoxadiazol-5-ylmethyl)-5′-fluoro-2H,5H-spiro[imidazolidine-4,3′-indole]-2,2′,5(1′H)-trione,
5′-chloro-7′-methyl-1′-(2-phenylethyl)-2H,5H-spiro[imidazolidine-4,3′-indole]-2,2′,5 (1′H)-trione,
1′-(2,1,3-benzoxadiazol-5-ylmethyl)-5′-chloro-7′-methyl-2H,5H-spiro[imidazolidine-4,3′-indole]-2,2′,5(1′H)-trione,
5′-chloro-7′-methyl-1′-{[6-(trifluoromethyl)-3-pyridinyl]methyl}-2H,5H-spiro[imidazolidine-4,3′-indole]-2,2′,5(1′H)-trione,
5′-chloro-7′-methyl-1′-[(5-methyl-3-isoxazolyl)methyl]-2H,5H-spiro[imidazolidine-4,3′-indole]-2,2′,5(1′H)-trione,
5′-chloro-7′-methyl-1′-(2-{[2-(methyloxy)phenyl]oxy}ethyl)-2H,5H-spiro[imidazolidine-4,3′-indole]-2,2′,5(1′H)-trione,
5′-chloro-1′-[(2-chloro-1,3-thiazol-5-yl)methyl]-7′-methyl-2H,5H-spiro[imidazolidine-4,3′-indole]-2,2′,5(1′H)-trione,
1′-[(2,6-dichloro-4-pyridinyl)methyl]-5′-methyl-2H,5H-spiro[imidazolidine-4,3′-indole]-2,2′,5(1′H)-trione,
5′-methyl-1′-(tetrahydro-2H-pyran-2-ylmethyl)-2H,5H-spiro[imidazolidine-4,3′-indole]-2,2′,5(1′H)-trione,
5′-methyl-1′-(2-propynyl)-2H,5H-spiro[imidazolidine-4,3′-indole]-2,2′,5 (1′H)-trione,
1′-(imidazo[1,2-a]pyridin-2-ylmethyl)-5′-methyl-2H,5H-spiro[imidazolidine-4,3′-indole]-2,2′,5(1′H)-trione,
or a pharmaceutically-acceptable salt thereof.

33. A method of treating VR1 mediated disorders in a mammal in need thereof comprising administering to such mammal an effective amount of a compound of claim 27.

34. A method of treating acute pain disorders in a mammal, which comprises administering to a person in need thereof an effective amount of a compound of claim 27.

35. A method of treating chronic pain disorders in a mammal, which comprises administering to a person in need thereof an effective amount of a compound of claim 27.

36. A method of treating acute neuropathic pain disorders in a mammal, which comprises administering to a person in need thereof an effective amount of a compound of claim 27.

37. A method of treating chronic neuropathic pain disorders in a mammal, which comprises administering to a person in need thereof an effective amount of a compound of claim 27.

38. A method of treating acute inflammatory pain disorders in a mammal, which comprises administering to a person in need thereof an effective amount of a compound of claim 27.

39. A method of treating chronic inflammatory pain disorders in a mammal, which comprises administering to a person in need thereof an effective amount of a compound of claim 27.

40. A method of treating low back pain, post-operative pain, visceral pains like chronic pelvic pain, cystitis, including interstitial cystitis and pain related thereto, ischeamic, sciatia, diabetic neuropathy, multiple sclerosis, arthritis, fibromyalgia, pain and other signs and symptoms associated with psoriasis, pain and other signs and symptoms associated with cancer, emesis, urinary incontinence, hyperactive bladder, HIV neuropathy, gastro-esophageal reflux disease (GERD), irritable bowel syndrome (IBS), inflammatory bowel disease (IBD) and/or pancreatitis, in a mammal, which comprises administering to a person in need thereof an effective amount of a compound of claim 27.

41. A method of treating osteoarthritis, rheumatoid arthritis, asthma, cough, chronic obstructive lung disease, specifically chronic obstructive pulmonary disease (COPD) and emphysema, lung fibrosis, and interstitial lung disease in a mammal, which comprises administering to a person in need thereof an effective amount of a compound of claim 27.

42. A method of treating respiratory diseases in a mammal, which comprises administering to a person in need thereof an effective amount of a compound of claim 27.

43. A method of treating obesity in a mammal, which comprises administering to a person in need thereof an effective amount of a compound of claim 27.

44. A method of treating migraine in a mammal, which comprises administering to a person in need thereof an effective amount of a compound of claim 27.

45. A method of treating burn induced pain and/or inflammatory pain resulting from burn injuries in a mammal, which comprises administering to a person in need thereof an effective amount of a compound of claim 27.

46. A pharmaceutical composition comprising as active ingredient a therapeutically effective amount of a compound according to claim 27, in association with one or more pharmaceutically acceptable diluents, excipients and/or inert carriers.

47. The pharmaceutical composition according to claim 46 for use in treatment of VR1 mediated disorders and for treatment of acute and chronic pain disorders, acute and chronic neuropathic pain and acute and chronic inflammatory pain, and respiratory diseases.

48. A process for the preparation of compounds of formula I, wherein R¹ to R⁹ and X are as defined as in claim 27, comprising:

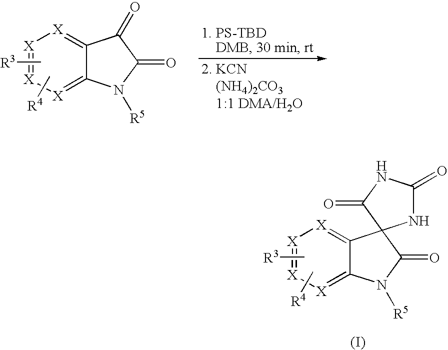

49. Compounds selected from:
(2E)-3-(3,4-dichlorophenyl)prop-2-en-1-ol,
1,2-dichloro-4-[(1E)-3-chloroprop-1-en-1-yl]benzene,
1-[(2E)-3-(3,4-dichlorophenyl)prop-2-en-1-yl]-5-(trifluoromethoxy)-1H-indole-2,3-dione,
tert-butyl (2,3-dioxo-2,3-dihydro-1H-indol-1-yl)acetate,
tert-butyl (2,2′,5-trioxospiro[imidazolidine-4,3′-indol]-1′(2′H)-yl)acetate,
(2,2′,5-trioxospiro[imidazolidine-4,3′-indol]-1′(2′H)-yl)acetic acid,
[2-(3,4-dichlorophenyl)cyclopropyl]methanol,
1,2-dichloro-4-[2-(chloromethyl)cyclopropyl]benzene,
1-{[2-(3,4-dichlorophenyl)cyclopropyl]methyl}-1H-indole-2,3-dione,
1-[(2E)-3-(3,4-dichlorophenyl)prop-2-en-1-yl]-1H-indole-2,3-dione, and
1′-[(2E)-3-(3,4-dichlorophenyl)prop-2-en-1-yl]-2H,5H-spiro[imidazolidine-4,3′-indole]-2,2′,5(1′H)-trione,
or pharmaceutically-acceptable salts thereof.

50. Compounds selected from:
1-[(2E)-3-(3,4-dichlorophenyl)prop-2-en-1-yl]-1H-indole-2,3-dione,
1-{[2-(3,4-dichlorophenyl)cyclopropyl]methyl}-1H-indole-2,3-dione, and
(2,2′,5-trioxospiro[imidazolidine-4,3′-indol]-1′(2′H)-yl)acetic acid.
or pharmaceutically-acceptable salts thereof.

51. The use of compounds according to claim 49 as intermediates in the preparation of the compound of formula I.
